(19) United States
(12) Patent Application Publication
(10) Pub. No.: US 2004/0009937 A1
(43) Pub. Date: Jan. 15, 2004

(54) Methods and composition for delivering nucleic acids and/or proteins to the respiratory system

(76) Inventors: Wei Chen, San Diego, CA (US); Xiaoli Fu, Carlsbad, CA (US); Sherry Nouraini, Vista, CA (US); Zhiqing Zhang, Carlsbad, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/353,137

(22) Filed: Jan. 27, 2003

Related U.S. Application Data
(63) Continuation-in-part of application No. 10/280,769, filed on Oct. 25, 2002.
(60) Provisional application No. 60/401,465, filed on Aug. 5, 2002.
(60) Provisional application No. 60/353,885, filed on Jan. 31, 2002.
(60) Provisional application No. 60/353,923, filed on Jan. 31, 2002.
(60) Provisional application No. 60/353,964, filed on Jan. 31, 2002.

(51) Int. Cl. A61K 48/00; A61L 9/04; A61K 9/14

(52) U.S. Cl. 514/44; 424/46

(57) ABSTRACT

Methods and compostions related to the fields of bacteriology, immunology and gene therapy are provided. In general modified microflora for the delivery of vaccines, allergens and therapeutics to the mucosal surfaces of the respiratory tract are provided. In particular, the compositions and methods are directed at inducing an M-cell mediated immune response to pathogenic diseases. Specifically, methods of vaccine preparation, delivery and mucosal immunization using a Lactic Acid Bacteria (LAB), yeast and LAB that have been modified through fusion with E. coli to either present on its cell surface, or secrete, antigenic epitopes derived from pathogenic microorganisms and/or to secrete a therapeutic protein sequence are disclosed.

### RELATED APPLICATIONS

This application claims priority to provisional application serial Nos. 60/401,465 filed Aug. 5, 2002, 60/353,885 filed Jan. 31, 2002, 60/353,923 filed Jan. 31, 2002, and 60/353,964 filed Jan. 31, 2002 and is a continuation-in-part of co-pending U.S. patent application Ser. No. 10/280,769 filed Oct. 25, 2002 the contents of which are incorporated herein by reference in their entirety.

### FIELD OF THE INVENTION

The present invention relates to the fields of bacteriology, immunology and gene therapy. In general, this invention relates to the use of modified microflora for the delivery of vaccines, allergens and therapeutics to the mucosal surfaces of the respiratory tract. In particular, this invention provides novel compositions and methods for inducing an M-cell mediated immune response to pathogenic diseases. Specifically, this invention relates to a method of vaccine preparation, delivery and mucosal immunization using a Lactic Acid Bacteria (LAB), yeast and LAB that have been modified through fusion with E. coli to either present on its cell surface, or secrete, antigenic epitopes derived from pathogenic microorganisms and/or to secrete a therapeutic protein sequence.

Referecnes

Various publications or patents are referred to in parentheses throughout this application to describe the state of the art to which the invention pertains. Each of these publications or patents is incorporated by reference herein. Complete citations of scientific publications are set forth in the text or at the end of the specification.

### BACKGROUND OF THE INVENTION

There are at least two immune systems, a “peripheral” or “systemic” and a “mucosal” immune system (Ogra et al., 1994). These systems operate both separately and simultaneously but may interact with one another via specific lymphocytic modulators to mount an effective immune response. The determining factor for which immune response will react first is the way in which pathological antigens are acquired by the individual and processed by the various lymphatic tissues.

Mounting an effective immune response depends upon the continuous movement of lymphocyte associated cells through blood, tissue and lymph (Anderson and Shaw, 1996). Lymphoid cells travel to the secondary lymphoid organs of the spleen, lymph nodes and to specialized mucosal tissue called Peyer's patches to encounter antigens acquired from the environment via blood, lymph or across mucous membranes, respectfully. Where and by which cells antigens are presented to these trafficking lymphatic cells significantly influences the outcome of the immune response with respect to T cell activation and B cell conversion into a particular antibody isotope and future homing preference of memory and effector lymphoid cells.

Antigens in lymph are filtered, trapped, processed and presented where the lymph passes over fixed antigen-presenting cells in lymph nodes. This antigen presentation by lymph nodes primarily results in “peripheral” immunity and the conversion of appropriate B cells into the specific IgG or IgM antibody. Antigens in blood are presented at specific blood/tissue interfaces in the spleen, which also primarily results in evoking “peripheral” immunity, however, due to the spleen's function of accommodating both antigen-presenting cells and activated T-and B cells from various other tissues, it is possible that cross talk between the two systems may amount to either peripheral or mucosal immunity or both. Antigens in the lumens of enteric organs (i.e., the respiratory and gastrointestinal tracts) are non-destructively endocytosed by specialized epithelial cells called “M” cells and transcytosed onto lymphoid cells in the Peyer's patches where response to antigen presentation primarily triggers commitment to “mucosal” immunity and the release of specific IgA antibodies into mucosal secretions.

The spaces inside the nose, throat, lungs and gut are continuous with the outside world, exposing these tissues to toxic and pathogenic threats from the environment. For protection, the respiratory, gastrointestinal and urogenital tracts are composed of mucosal surfaces made of a layer of mucus coated epithelial cells, joined cell to cell by gasketlike intercellular tight junctions. Facing an environment rich in microflora, these mucosal surfaces present a cellular barrier that is the first interface between pathogens and host. Thus they are critical in the prevention of infectious diseases.

The epithelial linings of the oral cavity, pharynx and esophagus are lined by a multi-layered squamous epithelia while the mucosal surfaces of the upper respiratory tract are predominantly lined by a single layer of simple epithelial cells. In the upper respiratory tract, the epithelial cells of the lungs are well equipped to face such a pathogen-rich foreign environment. This vast cellular barrier consists of a delicate monolayer of cells actively engaged in absorption of air and it is generally able to exclude potentially harmful and antigenic materials.

Within this mucosal epithelial lining of the respiratory tract, bits of lymphoid tissue make up the organized mucosa lymphoid follicle-associated epithelium (FAE) tissue. Though the epithelium that lines the respiratory tract is impermeable to macromolecules and microorganisms, in mucosal inductive sites, such as the Peyers patches in the upper respiratory tract, the lymphoid FAE contains microfold, or M cells, that allow the transportation of antigens and microorganisms, for antigen sampling. M cells, in simple epithelia only occur over organized lymphoid follicles. Hence, at FAE sites, rich in M cells, there is a highly developed collaboration of the specialized epithelia with antigen-presenting and lymphoid cells. Through active transepithelial vesicular transport, M cells transport macromolecules, particles, and microorganisms from the lumen, across their cytoplasm and directly into the intraepithelial mucosal lymphoid follicles and to organized mucosal lymphoid tissues that are designed to process antigens and initiate a mucosal immune response that results in secretory immunity—the process by which mucosal surfaces of the lung are bathed with protective antibodies.

Hence, M cells provide local, functional openings in the epithelial barrier through which vesicular transport occurs. Restriction of M cells to the sites directly over lymphoid follicles (FAE) serves to reduce the inherent risk of transporting foreign material and microbes across the epithelial barrier by assuring immediate exposure to phagocytes and antigen-presenting cells. The apical surfaces of M cells, facing the lumen, are distinguished from neighboring cells by the absence of a typical brush border and the presence of variable microvilli or microfolds with large intermicrovillar endocytic domains. A basal invagination in M cells creates a unique feature of the M cell, which is an intraepithelial “pocket” or space that both shortens the distance that transcytotic vesicles must travel from the apical to the basolateral surface and provides a docking site for lymphocytes, such as B and CD4 T cells, macrophages and dendritic cells to gather. M cells also have basal processes that extend into the underlying lymphoid tissue where they make direct contact with lymphoid and/or antigen-presenting cells, which likely plays a role in the presentation of antigens after M cell transport.

M cells engage in several different modes of transcytosis for the transport of foreign material into endosomal tubules, vesicles and large multivesicular bodies in the M cell apical cytoplasm and to their subsequent release by exocytosis into the pocket. Adherent viruses and macromolecules are taken up by adsorptive endocytosis via clathrin-coated pits and vesicles. Non-adherent materials are taken up by fluid-phase endocytosis in either coated or uncoated vesicles. Large adherent particles and bacteria trigger phagocytosis, involving the extension of cellular processes and the reorganization of the submembrane actin network.

The ability of M cells to conduct transport of intact macromolecules from one side of the barrier to the other involves the directed movement of membrane vesicles. Although the molecular mechanisms of this transport have not been determined in M cells, it is safe to assume that the membrane traffic conducted by M cells depends on the polarized organization and signaling networks typical of polarized epithelial cells. M cells are unique among epithelial cells in that transepithelial vesicular transport is the major pathway for endocytosed materials. Studies have shown that endocytic vesicles formed at the, apical surface of M cells first deliver their cargo to endosomes in the apical cytoplasm and that these acidify their content and contain proteases.

One of the primary components in the M cell pocket is B cells. The B cells in the pocket express IgM but not IgG or IgA, suggesting that B memory cells and/or initial B cell differentiation may occur here. The presence of memory phenotypes suggests that cells in the pocket have positioned themselves for re-exposure to incoming antigens. It has been suggested that B lymphoblast traffic into the M cell pocket may allow for repeated antigen exposure and extension and diversification of the immune response. However, immediately under the FAE, there is an abundance of other B lymphoblasts, helper T cells, and antigen-presenting cells that are sufficient for initiating an immune response.

Lumenal antigens transcytosed by M cells are immediately delivered to these antigen-processing and -presenting cells that then migrate to antigen-specific lymphocytes, in the underlying lymphoid follicles located in the nasal-associated lymphoid tissue (NALT), which further induces their proliferation. Thus, passage of antigens and microorganisms through M cells is an essential step for the development of mucosal immune responses. This process results in the development of IgA-producing B cells, some of which move into the vasculature and then back to the mucosal surfaces, efficiently seeding specific mucosal immunity.

The first step in the induction of a mucosal immune response is the transport of antigens across the epithelial barrier. Following antigen processing and presentation in inductive sites, IgA-committed, antigen-specific B lymphoblasts proliferate locally and then migrate via the bloodstream to local and distant mucosal and secretory tissues. There they differentiate primarily into polymeric IgA-producing plasma cells, which are important components of NALT, and are transported across epithelial cells into glandular and mucosal secretions via receptor-mediated transcytosis.

Hence, mucosal immunity forms a first line of defense against mucosally transmitted pathogens such as influenza and is important for long-term protection. Mucosal defense against pathogens consists of both innate barriers, such as mucous, epithelium, and innate immune mechanisms, and adaptive host immunity, which at mucosal surfaces consists predominantly of CD4⁺ T cells, secretory immunoglobulin A (S-IgA), and antigen-specific cytotoxic T-lymphocytes (CTLs). Under healthy circumstances, transport by M cells and the resulting secretion of antimicrobial sIgA antibodies limit the intensity or duration of mucosal disease and prevent reinfection.

The principal antibody involved in mucosal immunity is secretory immunoglobulin A (S-IgA). Its production is the hallmark of the mucosal immune system and it provides an important first line of defense against invasion of deeper tissues by pathogens (Underdown and Mestecky, 1994). The antibodies of the mucosal immune system function outside the body at luminal surfaces of the moist epithelium lining conjunctiva, nasopharynx, oropharynx, gastrointestinal, respiratory and urogenital tracts and in the ducts or acini of exocrine glands. Hence, this class of antibody requires the cooperation of two cell types for optimal activity. One cell makes the IgA and another cell transports it to the lumen of the respiratory system where it works.

S-IgA results from transcytosis of pIgR across the epithelium through binding to the pIgR (receptor). Secretory IgA is produced by lamina propria B plasma cells and is transported into the lumen by crypt epithelial cells throughout the gut. The antibody-forming plasma cell releases dimeric IgA, which is postranslationally associated with the J chain. The J chain holds the two polyIgA molecules together and facilitates binding to the poly-Ig receptor (pIgR) displayed on the abluminal side of epithelial cells. This complex is transported in endosomes to the luminal side of the epithelial cell and released into the secretions. The portion of the poly-Ig receptor retained with secreted IgA is called the secretory component. S-IgA is released from the pIgR by cleavage of the receptor, resulting in pIgR covalently associated with a substantial part of the pIgR, i.e., the secretory component. Once secreted into the lumen, IgA does not adhere to the apical surfaces of enterocytes but adheres selectively to the apical membranes of M cells.

Antigenic exposure at mucosal sites, further activates mucosal B and T-lymphocytes to emigrate from the inductive site and home to various mucosal effector sites. The common mucosal immune system involves homing of antigen-specific lymphocytes to mucosal effector sites other than the site where initial antigen exposure occurred. This pathway has almost exclusively been documented for S-IgA antibody responses at mucosal surfaces mediated by B cells, but similar events take place with T cells.

Another essential component found in the M cell pocket are T cells. T-cells express either T-helper 1 (Th1) or T-helper 2 (Th2) cytokines. T-cell helper functions play important roles in generating antigen-specific humoral and cell-mediated immunity in both systemic and mucosal compartments. Cytokines drive the differentiation of T-helper 0 (Th0) cells into either T-helper 1 (Th1) or T-helper 2 (Th2). The differentiation of Th0 cells into either Th1 or Th2 is driven by cytokines such as interleukin 12 (IL-12), interferon g (IFN-g), and IL-4, respectively. For example, intracellular pathogens, such as viruses and intracellular bacteria, induce production of IL-12 by activated macrophages, which induces IFN-g production in natural killer (NK) cells, and in turn drives the differentiation of Th0 cells toward the Th1 phenotype and the induction of the cell mediated immune response.

The Th1-type responses are associated with cell-mediated immunity, such as delayed-type hypersensitivity and IgG2a antibody responses. When Th1 responses are preponderant (as they are in skin-draining lymph nodes), T helper cells secrete IL 2, IL-12 and IF gamma, resulting in selective expression of IgG immunoglobulins and activation of cytotoxic T cells and armed mononuclear phagocytes (Weinstein et al., 1991; Kang et al., 1996; and Ariizumi et al., 1995).

Where Th2 responses are preponderant (as they are in mucosal sites), T helper cells secrete IL 4, 5, 6, 10, etc, resulting in selective expression of different immunoglobulin isotopes including IgA (Hiroi et al., 1995; Lebman and Coffinan, 1994). In mucosal sites, abundance of the cytokine TGF beta-1 programs Th0 cells to develop into Th2 cells (Lebman and Coffman, 1994; Young et al., 1994). The cytokines secreted by Th2 cells contribute to expansion and differentiation of B cells committed to IgA expression. TGF beta-1 also contributes to selective expression of IgA antibodies by favoring immunoglobulin heavy chain gene switching to IgA, and by suppressing expression of other isotopes (Lebman and Coffinan, 1994; Stavnezer, 1995). TGF beta-1 is not widely distributed in peripheral lymph nodes where there is selective expression of Th1 cellular responses and IgG antibodies. When an exogenous antigen is encountered, Th0 cells differentiate into Th2 cells, triggering CD4⁺ T cells to produce IL-4. IL-4 induces the conversion of more Th0 into Th2 cells at the same time as inducing already converted Th2 cells to produce and secrete more IL-4, expanding Th2-cells, which support the associated immune response. The production of IL-4, then goes on to support IgG and IgE as well as IgA production.

Although S-IgA the predominant effector molecule that protects mucosal surfaces, the peripheral cellular immune system eventually begins to play an important role. The strategic advantage of cell-mediated versus antibody-mediated immune responses is that T cells can recognize peptides derived from core proteins of the pathogen, such as influenza virus. Core proteins are usually expressed and presented much earlier during infection than proteins targeted for neutralizing antibodies. Subsequently, cell-mediated immunity (CMI) occurs before the induction of antibodies and forms an early line of defense; although antibodies to core proteins are also formed later in the immune response. Besides supporting humoral immunity, CD4⁺ T-helper cells function in CMI as producers of cytokines, which mediate delayed-type hypersensitivity and support CTLs. For example, major histocompatibility complex (MHC)-restricted CTL responses are supported by Th1 cells.

Mucosal infection by intracellular pathogens eventually results in the induction of cell-mediated immunity, as manifested by CD4-positive (CD4⁺) T helper-type 1 cells, as well as CD8⁺ cytotoxic T-lymphocytes (CTLs). T lymphocytes involved in peripheral and mucosal cellmediated immunity segregate into functional subclasses (Punt and Singer, 1996). T-helper cells (CD-4) and cytotoxic T-lymphocytes (CD-8) both assume immuno-regulatory roles during immune responses. They may also differentiate into the various effector cells that control the varied traffic patterns and functions of the immune response (Salgame, 1991; Anderson and Shaw, 1996; Ebnet et al.). As expected, it is the T cell's cytokine secretions that direct immunoreactive cell commitment to either peripheral or mucosal immune functions.

CTLs play an important role in the elimination of cells infected with various intracellular pathogens by recognizing pathogen-specific antigen/MHC complexes. Antigen-specific CTLs inhibit further spread of pathogens and help to terminate infections. Compartmentalization of pathogen-specific CTL responses has been reported and located at the site of initial infection. For example, CTLs preferentially compartmentalize in mucosa-associated lymphoreticular tissues after pulmonary or intestinal infection. The presence of CTLs in mucosal compartments may contribute to the control of, and recovery from, infection by intracellular pathogens at mucosal surfaces. Since different pathogens have distinct infection routes or different localization in the host, compartmentalization of protective, antigen-specific CTLs may vary, based on the specific pathogen. In general, however, mucosal infection induces primarily antigen-specific CTLs in the mucosal compartment and mucosa-associated lymphoid organs and depends on mucosal infection to control pathogens at the port of entry, i.e., the mucosal surfaces. These responses normally occur shortly after the synthesis of secretory immunoglobulin A (S-IgA) antibodies.

A common procedure to help fight infectious diseases involves immunization. Generally, immunization involves priming the immune system to swiftly destroy specific disease-causing agents, or pathogens, before the agents can multiply enough to cause symptoms. Classically, this priming has been achieved by presenting the immune system with a vaccine that contains either whole viruses or bacteria that have been killed or made too weak to proliferate much. On detecting the presence of a foreign organism in a vaccine, the immune system behaves as if the body were under attack by a fully potent antagonist. It mobilizes its various forces to root out and destroy the apparent invader-targeting the campaign to specific antigens (proteins recognized as foreign).

Parenteral immunization is the most common route of vaccination. It usually elicits a peripheral acute immune response, with protective IgM/IgG antibodies and peripheral cell-mediated immunity. The acute response soon abates, but it leaves behind sentries, known as “memory” cells, that remain on alert, ready to unleash whole armies of defenders if the real pathogen ever finds its way back into the body. Effective as they are, injected vaccines initially bypass mucous membranes and usually fail to stimulate mucosal lymphatic tissues to generate protective IgA antibodies and therefore they fail to stimulate mucosal immunity.

This presents a problem because many hazardous agents that spread through the systemic circulation, initially infect across the mucosae, entering the body through the nose, mouth or other openings. Hence, the first defenses they encounter are those in the mucous membranes that line the airways, the digestive tract and the reproductive tract; these membranes constitute the biggest pathogen-deterring surface in the body. Protection against these agents requires vaccines that not only induce a peripheral but also a mucosal immune response. As stated above, when the mucosal immune response is initiated, it generates IgA antibodies that dash into the cavities of those passageways, neutralizing any pathogens they find. An effective reaction also activates a systemic response, in which circulating cells of the immune system help to destroy invaders at distant sites.

Another complication with respect to “paranteral” vaccination is that classic vaccines pose a risk that the vaccine microorganisms will somehow spring back to life, causing the diseases they were meant to forestall.

Because of this complication alternative approaches to traditional modes of vaccination are being sought. One of these is the use of DNA vaccines, wherein a plasmid containing a DNA segment from a pathogenic organism is administered to induce protection against various pathogens, including hepatitis B virus, herpes simplex virus, MV, malaria and influenza.

The methods currently under development with respect to DNA vaccines, are also plagued with problems. First of all, delivery is complicated. The gene or cDNA needs to be incorporated into an appropriate expression vector and delivered into an appropriate protein-synthesizing organism (e.g., E. coli, S. cerevisiae, P. pastoris, or other bacterial, yeast, insect, or mammalian cell) for the production of multiple copies of the gene of interest. Further, the DNA must be isolated, put into another expression system and delivered into a host, where the gene, under the control of an endogenous or exogenous promoter, can be appropriately transcribed and translated. The use of multiple expression vectors (including, but not limited to, phage, cosmid, viral, and plasmid vectors) are expensive, difficult to make, and hard to administer. Further, effective administration often requires the co-administration of viral elements for delivery into the host, which carries the risk of recombinant competent retrovirus formation.

Another method for inducing immunal protection provides the administration of subunit vaccine preparations, composed primarily of antigenic proteins divorced from a pathogen's genes. By themselves, these proteins have no way of establishing an infection. However, induction of antibodies and CTL in the systemic but not the mucosal compartment normally results, further these vaccines are expensive to produce, purify and maintain.

A further problem related to traditional modes of vaccination is that physiological changes in the human host may be contributing to the emergence of new diseases. Perhaps emerging pathogens become resistant to antibiotics or (through genetic recombination) become more resistant to host defenses. Recombination events or lack of exposure can result in loss of immunity of the population to the pathogen, as has been well documented with influenza virus. Recombination events increase the infection rate by the emerging pathogen and, in the case of influenza virus, occasionally result in pandemics.

Hence, despite advances in disease prevention and immunization, new and reemerging infectious diseases are tipping the balance in favor of the parasite; systemic immunization is important but continued development of mucosal vaccines will be needed to effectively combat these new threats. For this reason, oral vaccines are currently being developed. They are better at evoking both a “mucosal” and a “peripheral” immune response, more cost effective and they are more convenient than. vaccines of the more commonly used parenteral delivery system. Currently, the oral vaccines being developed tend to focus on the development and utilization of modified pathogenic organisms, such as Salmonella species, as antigen carvers for oral immunization (Stocker, U.S. Pat. No. 4,837,151, Auxotrophic Mutants of Several Strains of Salmonella; Clements et al., U.S. Pat. No. 5,079,165, Avirulent Strains of Salmonella; Charles et al., U.S. Pat. No. 5,547,664, Live-attenuated Salmonella). However, even when these pathogens are attenuated they may pose a danger of reverting to pathogenicity and being harmful to the host animal.

The present inventor has been researching the possibility of using Lactic acid bacteria (LAB) as a live vehicle for the production and delivery of therapeutic molecules such as antigens. The lactic acid bacteria (LAB) constitute a family of gram-positive bacteria that are well known for their use in industrial food fermentations and for their probiotic properties. LAB, in general, and Lactococcus lactis and Streptococcus thermophilus in particular, possess certain properties that make them attractive candidates for use in oral vaccination. These properties include adjuvant activity, mucosal adhesive properties and low intrinsic immunogenicity.

Given the problems inherent in parenteral vaccination, especially as they relate to DNA or sub-unit vaccines, the current inventor has developed novel compositions and methods of using non-pathogenic Lactococcus and Streptococcus bacteria for the delivery of both antigens and therapeutics to the upper respiratory tract for the purposes of vaccination and/or gene therapy.

One species of particular note are Lactococcus lactis. They are low GC count, rod-shaped bacteria that are critical for manufacturing dairy products like buttermilk, yogurt, cheese, pickled vegetables, beer, wine, breads and other fermented foods. The L. lactis genome contains six prophages (carrying nearly 300 genes or ca. 14% of the total coding capacity) and 43 insertion elements. Sequence data has revealed a low number of two-component signal transducers and very few sigma. Genome analysis also confirms the total lack of genes and enzymes involved in the citric acid cycle although the bacteria still maintains the functions necessary for aerobic respiration. Another bacteria of particular note, and of use in food grade fermentation processes such as that used to make cheese, is Streptococcus thermophilus.

With respect to Lactic Acid Bacteria in general, several procedures already exist for the creation of LAB transformants. Leer et al. (WO095/35389) disclose a method for introducing nucleic acid into microorganisms, including microorganisms such as Lactobacillus and Bifidobacterium. The method of Leer et al. is based on limited autolysis before the transformation process is undertaken. Published PCT application PCT/NL96/00409 discloses methods for screening non-pathogenic bacteria, in particular LAB of the genera Lactobacillus and Bifidobacterium, for the ability to adhere to specific mucosal receptors. An expression vector is also disclosed that comprises an expression promoter sequence, a nucleic acid sequence, and sequences permitting ribosome recognition and translation capability. This reference indicates that various strains of Lactobacillus can be transformed so as to express heterologous gene products including proteins of pathogenic bacteria. Further, oral administration of recombinant L. lactis has been used to elicit local IgA and/or serum IgG antibody responses to an expressed antigen. Wells et al, Mol. Microbiol. 8: 1155-1162, 1993. In addition, Casas et al. (U.S. Pat. No. 6,100,388) discloses that L. reuteri, can be transformed with heterologous DNA, and can express the foreign protein on the cell surface or secrete it, while EP 1084709 A1 discloses that L. plantarum can, as well, be transformed to express an antigenic fragment either intracellularly or on the cell surface. See also See U.S. Pat. Nos. 5,149,532 and 6,100,388.

These references all disclose the use of certain species of bacteria for use in vaccination. The methods therein described are altogether time consuming, expensive and inefficient. Furthermore, the above cited references primarily target the gastrointestinal tract, passing over the various mucosal surfaces of the respiratory tract.

In addition, with respect to the methods currently practiced, different expression systems can be required for each specific species sought to be used for antigen delivery. Appropriate promoters, enhancers and selectable markers often have to be developed. Several different transformations may need to take place to determine a viable system so as to ensure appropriate expression levels in vitro and in vivo. All of this adds both tremendous time and cost. What is needed is a system whereby previously known and commercially available expression systems may be used to express heterologous protein elements in commercially available, safe, Lactic Acid Bacteria for the delivery of antigens and/or therapeutics to the respiratory tract. Due to the work of the present inventor such a system is hereby presented.

### SUMMARY OF THE INVENTION

Due to the complications inherent in parenteral immunization, specifically, its failure to evoke a muscosal immune response, its lack of protection against pathogenic agents that initially infect across the mucosae, and the risk that attenuated live vaccine microorganisms mutate into their pathogenic forms, the present inventor has turned to developing an alternative approach to disease prevention, an approach that will be effective both for vaccination and gene therapy.

The biggest hurdle preventing the successful development of DNA or protein based compositions and methods for the treatment of malignant conditions is that of delivery. Where the target is the respiratory system, delivery is extremely complicated and inefficient. The methods currently being studied involve either the delivery of naked DNA/lyposome conjugates, which suffers from a low transduction rate, or delivery involves the development of disease specific expression vectors that are host specific and are difficult to produce, maintain and administer. Further, the delivery mechanism often sought involves the use of viral elements carrying with it the risk of recombinant competent retrovirus formation.

As stated above, a promising theory for inducing both a mucosal and systemic immune response, being pursued by the present inventor, involves the administration of mucosal vaccines delivered by live microflora organisms, including bacteria and yeast, to the respiratory tract. Evidence indicates the existence of a common M cell mediated pathway for inducing both mucosal and systemic (cell-mediated) immunity via the nasal-associated lymphoid tissue (NALT) in the upper respiratory system. Unlike normal CTL activity, which requires the migration of CTLs from distant sites to the systemic compartment before being primed, Antigen-specific CTL responses at mucosal surfaces associated with NALT are dictated by the induction of CTL locally.

Hence, antigen-specific CTL within the M cell pocket allow for quick, protective responses at any mucosal site-this concept has major implications for enhanced vaccine development. Since, mucosal antigen-specific memory CTL responses are observed primarily after mucosal immunization, optimal protection against pathogens requires the use of mucosal vaccines, especially in light of the recent discovery that an antigen-specific mucosal CTL response can induce systemic CTL and generate systemic immunization. Mucosal vaccines, when delivered by microflora, should come into contact with the lining of the respiratory tract and activate both mucosal and systemic immunity.

Other mechanisms being studied using microflora for vaccination purposes primarily target delivery to the gut. What is needed is a general mechanism that can be used across the board regardless of the biologically compatible microflora being used, a mechanism that will target delivery specifically to the mucosal immune inductive cells and allow for efficient, non-invasive and safe delivery to the respiratory tract.

The present inventor has developed novel compositions and methods for delivering both antigenic fragments and therapeutics to the respiratory tract using modified yeast and LAB (microflora).

Examples of suitable microflora for use in accordance with the teachings of the present invention inlcude, without limitation, members of the genus Lactobacillus, Lactococcus, Streptococcus and Saccharomyces. Furthermore, the microflora of the present invention have M cell binding elements for targeting of the bacteria to the mucosal surfaces of the respiratory tract.

The present invention also includes microflora that express antigens on the cell surface and/or secrete them. In this instance the antigen to be delivered should be coded for in concert with an appropriate modified secretion signal as well as an appropriate anchor signal. While for therapeutic applications, where a polypeptide needs to be fully processed and secreted (transmembraned) in large quantities, a fully encoded secretion signal may be necessary.

In summary, the present inventor has developed novel compositions and methods for delivering antigenic fragments and/or genetic elements to mucosal cells of the respiratory tract, for the induction of a mucosal immune response, and/or the delivery of therapeutic elements for the purposes of gene therapy. Specifically, the invention pertains to the production of novel modified microflora that can be used as delivery vehicles for heterologous nucleic acids.

In one embodiment, the invention comprises microflora derived from fusing two different strains of bacteria, specifically, a modified E. coli with a Lactic Acid Bacteria, such as non-pathogenic Streptococcus bacteria. More particularly, the E. coli has been modified by being transformed with an expression vector capable of driving expression of a heterologous nucleic acid within a host organism, i.e., either E. coli or the LAB and E. coli fusant. More particularly still, the LAB used is Streptococcus thermophilus or Lactococcus lactis.

In another embodiment the invention comprises microflora derived from yeast.

In yet another embidment of the present invetion the vaccine is comprised of microflora bacteria such as LAB.

The heterologous nucleic acid may encode for an antigen capable of being expressed on the cell surface of the microflora or secreted into the extracellular milieu of the respiratory system. Specifically, the antigenic element may be tumor, bacterial or viral antigens. Bacterial antigens that may be encoded may include, but not hereby limited to, Mycobacterium leprae antigens; Mycobacterium tuberculosis antigens; Rickettsia antigens; Chlamydia antigens; Coxiella antigens; malaria sporozoite and merozoite proteins, such as the circumsporozoite protein from Plasmodium berghei sporozoites; diphtheria toxoids; tetanus toxoids; Clostridium antigens; Leishmania antigens; Salmonella antigens; E. coli antigens; Listeria antigens; Borrelia antigens, including the OspA and OspB antigens; Franciscella antigens; Yersinia antigens; Mycobacterium africanum antigens; Mycobacterium intracellular antigens; Mycrobacterium avium antigens; Shigella antigens; Neisseria antigens; Staphylococcus, Helicobacter, peudomona, Treponema antigens; Schistosome antigens; Filaria antigens; Pertussis antigens; Staphylococcus antigens; Anthrax toxin, Pertussis toxin, Clostridium; Hemophilus antigens; Salmonella; Streptococcus antigens, including the M protein of S. pyogenes and pneumococcus antigens such as Streptococcus pneumoniae antigens.

Viral antigens that may be encoded may include, but not hereby limited to, mumps virus antigens; hepatitis virus a.b.c.d.e. HBV antigens; rabies antigens; polio virus antigens; Rift Valley Fever virus antigens; dengue virus antigens; measles virus antigens; rotavirus antigens; Human Immunodeficiency Virus (HIV) antigens, including the gag, pol, and env proteins as well as gp 120 and gp 160 of the HIV env; respiratory syncytial virus (RSV) antigens; Herpes virus antigens; parainfluenza virus antigens; measles virus antigens; snake venom antigens; human tumor antigens; Vibrio cholera antigens, as well as antigens from HCV, HAV, HPV, TB, Herpes, rubella, influenza, mumps, poliomyelitis, rotavirus, surface glycoprotein of malaria parasite, parvovirus, Epstein barr virus, poxvirus, rabies virus, pneumonia, cancer antigens like CEA and other similar antigenic fragments.

Furthermore, in an alternative particular embodiment the heterologous nucleic acid may code for a therapeutic protein capable of being expressed on the cell surface of the microflora or secreted into the extracellular milieu and to be delivered to the mucosal cells of the respiratory system. Specifically, the therapeutic element may be a gene of interest coding for insulin, growth hormone, Epogen, interferon, cytokines, interleukine, human albumin, activase, vitamins, anticancer agents taxol, factor VIII and IX; cancer antigens, whole antibodies, antibody fragments, antibiotics, hormones, pheromones, other small molecules like calcitonin.

The invention further encompasses the method of producing the modified microflora ands compositions containing these organisms. Moreover, the present invention includes related methods for using the modified microflora for treating, palliating or preventing diseases including diseases associated with various protein deficiency disorders such as Diabetes, Hemophilia, growth hormone deficiency, etc. as well as viral and bacterial infections such as AIDS, Hepatitis, Malaria, plague, smallpox, herpes, human papilloma virus, and rotavirus. Moroeover, the present invention can also be used to administer vaccines and immunotherapeutics for the treatment, palliation or prevention of cancer, including colon, lung, prostate, and the like.

In one embodiment the heterologous nucleic acid is inserted into an already existing and/or commercially available expression system for E. coli, and the E. coli bacteria is then fused with an LAB. The resultant fusant may then be associated with an appropriate biological carrier for the delivery of the LAB delivery vehicle to the respiratory system where the appropriate antigenic or therapeutic response may be induced. In a particular embodiment, the composition containing the fusant strain may be formulated so as to be administered intranasally for the purposes of inducing M cell mediated immunity (i.e., mucosal vaccination) and/or for the treatment of abhorrent conditions caused by a defect in normal protein production.

In another embodiment of the present invention microflora contains a construct coding for an M cell targeting factor. This factor may be included in the plasmid containing the heterologous nucleic acid to be inserted into the microflora, it may be on a separate plasmid therein, or inserted into the LAB-E. coli fusant surface membrane during regeneration of the outer membrane. Upon expression the M cell targeting factor allows the modified microflora to preferentially bind to M cells over other forms of epithelial cells. There are in general three types of elements which may be used to target M-cells (Chen et al. U.S. Pat. No. 6,060,082) (Ginkel et al. CDC. 6(2), 2000). One is lectin, which can be incorporated into a cell's surface. The second is the sigma protein from reovirus, which targets M cell factors and be expressed as a fusion protein. Wu, Y., et al., “M cell-targeted DNA vaccination” Proc. Natl Acad. Sci. USA 98(16): 9318-23 (2001). With regard to the sigma protein, one embodiment would be to encode the polynucleotide sequence for the protein on either the plasmid coding for the heterologous nucleic acid or on a sepate plasmid such that when the sequence is transcribed and the protein produced it is expressed it on the delivery host cell surface along with the antigenic or therapeutic protein to be expressed. The third method involves the development and use of monoclonal antibody fragments targeted specifically, or at least predominantly to M-cells. A further mechanism for targeting M cells, is by developing appropriate host strains, through mutation and selection that preferentially bind to epithelial cell in vitro, for instance, by using HeLa cells.

### BRIEF DESCRIPTION OF THE FIGURES

FIG. 1 depicts the expression of Green Fluorescent protein (GFP) on the surface of yeast cells transformed in accordance with the teachings of the present invention.

FIG. 2 graphically depicts the serological results from mice receiving an oral vaccine against influenza virus using the GPD plasmid versus controls.

FIG. 3 graphically depicts the serological results from mice receiving a subcutaneous vaccine influenza virus using the GPD plasmid versus controls.

FIG. 4 graphically depicts the serological results from mice receiving an oral vaccine against rotavirus VP7 using the GPD plasmid versus controls.

FIG. 5 graphically depicts the serological results from mice receiving a subcutaneous vaccine against rotavirus VP7 using the GPD plasmid versus controls.

FIG. 6 graphically depicts the serological results from mice receiving an oral vaccine against influenza virus using the pYD plasmid versus controls.

FIG. 7 graphically depicts the serological results from mice receiving a subcutaneous vaccine against influenza virus using the pYD plasmid versus controls.

FIG. 8 graphically depicts the serological results from mice receiving an oral vaccine against rotavirus VP7 using the pYD plasmid versus controls.

FIG. 9 graphically depicts the serological results from mice receiving a subcutaneous vaccine against rotavirus VP7 using the pYD plasmid versus controls.

FIG. 10 graphically depicts the serological results from mice receiving an intranasal vaccine against influenza virus using the pYD plasmid versus controls.

### DETAILED DESCRIPTION OF THE INVENTION

Introduction

In one embodiment the present invention modified microflora capable of expressing and or secreting a foreign protein formulated for intranasal delivery are provided. These modified microflora consists of either yeast or bacteria that are compatible with the mammalian body. In another embodiment of the present invention microflora bacteria are fused with a second type of bacteria that harbors an expression system capable of expressing a desired antigen or therapeutic protein.

Definitions

Various terms relating to the biological molecules of the present invention are used throughout the specification and claims. Prior to setting forth the invention, it may be helpful to an understanding thereof to setforth definitions of the terms that will be used hereinafter.

“Antigen” or “antigenic fragment,” immunoprotective epitope“or “epitope” refers to all or parts thereof of a protein or peptide capable of causing a cellular or humoral immune response in a subject (i.e., an animal or mammal). Such would also be reactive with antibodies from animals immunized with said protein. Furthermore, the terms “antigen,” “antigenic fragment” or “epitope” as used herein describing this invention, include any determinant responsible for the specific interaction with an antibody molecule. Antigenic or epitopic determinants usually consist of chemically active surface groupings of molecules such as amino acids or sugar side chains and have specific three-dimensional structural characteristics, as well as specific charge characteristics. Examples of antigens or epitopes that can be used in this invention include, but are not limited to, viral, bacterial, protozoan, microbial and tumor antigens.

An “antigenic or therapeutic element” may include, for example, antigenic or therapeutic DNA, cDNA, RNA, and antisense polynucleotide sequences.

A “coding sequence” or “coding region” refers to a nucleic acid molecule having sequence information necessary to produce a gene product, when the sequence is expressed.

The term “compatible” with reference to a mammalian body refers to the capability of co-existence, together in harmony, i.e., capable of being used in transfusion or grafting without immunological reaction.

The term “contacted” when applied to a cell is used herein to describe the process by which an antigen or therapeutic gene, protein or antisense sequence, and/or an accessory element, is delivered to a target cell, via a microflora delivery vehicle, or is placed in direct proximity with the target cell.

“Delivery of a therapeutic agent” may be carved out through a variety of means, such as by using oral delivery methods such as pill formulations or compositions formulated in such a way as to allow for oral administration, and the like. Such methods are known to those of skill in the art of drug delivery, however, preferable compositions include pharmaceutical formulations, comprising a antigenic or therapeutic gene, protein, or antisense polynucleotide sequence that may be delivered in combination with a microflora delivery vehicle, such as Lactobacillus or Saccharomyces. In such compositions, the gene may be in the form a DNA segment, plasmid, cosmid or recombinant vector that is capable of expressing the desired protein in a cell; specifically, a LAB-E. coli fusant cell. These compositions may be formulated for in vivo administration by dispersion in a pharmacologically acceptable grade of yogurt.

The term “expression cassette” refers to a nucleotide sequence that contains at least one coding sequence along with sequence elements that direct the initiation and termination of transcription. An expression cassette may include additional sequences, including, but not limited to promoters, enhancers, and sequences involved in post-transcriptional or post-translational processes.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. With respect to a protein, the term “heterologous” is herein understood to mean a protein at least a portion of which is not normally encoded within the chromosomal DNA of a given host cell. Examples of heterologous proteins include hybrid or fusion proteins comprising a bacterial portion and a eukaryotic portion, eukaryotic proteins being produced in prokaryotic hosts, and the like.

A “heterologous nucleic acid” is a DNA, cDNA or any form of RNA polynucleotide sequence, or hybrid thereof, as well as an amino acid sequence constituting a polypeptide, peptide fragment, or protein that is derived from a different species from the one in which it is being produced. Heterologous nucleic acid sequence may also include a nucleic acid sequence from the same species that is intended to replace or augment and endogenous nucleic acid sequence. This particularly true for gene therapy applications including gene replacement.

An “immunogenic composition” as used herein is an embodiment of the present invention that provides an antigen to an animal in a manner that facilitates the induction of an immune response. The immune response can be humoral or cellular or both and contains and immunogen, or a fragment or subunit thereos. Representative antigens include, but are not limited tumor antigens, viral antigens, parasitic antigens, fungal antigen and bacterial antigens. For example, bacterial antigens that may be encoded may include, but not hereby limited to, Mycobacterium leprae antigens; Mycobacterium tuberculosis antigens; Rickettsia antigens; Chlamydia antigens; Coxiella antigens; malaria sporozoite and merozoite proteins, such as the circumsporozoite protein from Plasmodium berghei sporozoites; diphtheria toxoids; tetanus toxoids; Clostridium antigens; Leishmania antigens; Salmonella antigens; E. coli antigens; Listeria antigens; Borrelia antigens, including the OspA and OspB antigens; Franciscella antigens; Yersinia antigens; Mycobacterium africanum antigens; Mycobacterium intracellular antigens; Mycrobacterium avium antigens; Shigella antigens; Neisseria antigens; Staphylococcus, Helicobacter, peudomona, Treponema antigens; Schistosome antigens; Filaria antigens; Pertussis antigens; Staphylococcus antigens; Anthrax toxin, Pertussis toxin, Clostridium; Hemophilus antigens; Salmonella; Streptococcus antigens, including the M protein of S. pyogenes and pneumococcus antigens such as Streptococcus pneumoniae antigens.

Viral antigens that may be encoded may include, but not hereby limited to, mumps virus antigens; hepatitis virus a.b.c.d.e. HBV antigens; rabies antigens; polio virus antigens; Rift Valley Fever virus antigens; dengue virus antigens; measles virus antigens; rotavirus antigens; Human Immunodeficiency Virus (HIV) antigens, including the gag, pol, and env proteins as well as gp 120 and gp 160 of the HIV env; respiratory syncytial virus (RSV) antigens; Herpes virus antigens; parainfluenza virus antigens; measles virus antigens; snake venom antigens; human tumor antigens; Vibrio cholera antigens, as well as antigens from HCV, HAV, HPV, TB, Herpes, rubella, influenza, mumps, poliomyelitis, rotavirus, surface glycoprotein of malaria parasite, parvovirus, Epstein barr virus, poxvirus, rabies virus, pneumonia, cancer antigens like CEA and other similar antigenic fragments. fragment.

“Lactic Acid Bacteria” or “LAB” generally refers to a family of Gram positive bacteria that ferment carbohydrates to produce lactic acid as a fmaI product. Lactic acid bacteria live in the oral cavities and the alimentary tract and are utilized for the manufacture of fermentative foods, such as kimchi, yogurt, etc. They are known to produce various antimicrobial compounds, such as organic acids, hydrogen peroxide, diacetyl and bacteriocins, and are known to play an important role in maintaining the entrails healthy condition by utilizing carbohydrates as an energy source to produce lactic acid and antibacterial materials which inhibit the growth of the harmful bacteria. Among the lactic bacteria are those of the genera Streptococcus, Enterococcus, Lactococcus, Lactobacillus, and Bifidobacterium. Representative examples of these lactic acid-producing bacteria include Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

“Lactobacillus” refers to a lactic acid bacteria of the genus Lactobacillus that has the following bacteriological properties: namely Gram positive, rod shape, non-mobility, negative catalase, facultative anaerobic properties, optimum growth temperature of 30.degree. to 40.degree. C., no growth at 15.degree. C. and formation of DL-lactic acid.

“Microflora” as used herein includes bacteria, yeast, bacteria-bacteria fusants and bacteria-yeast fusants.

The term “modified” refers generally to a process whereby basic or fundamental changes are made to a given organism or system to bring about a new orientation or formation to or to serve a new end. In one embodiment a “modified microflora organism” is one that has been transformed with an expression vector encoding for an antigenic or therapeutic polypeptide and wherein the “modified microflora” expresses the antigenic or therapeutic polypeptide either on its surface and/or secretes it.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene. The term “DNA construct” is also used to refer to a heterologous region, particularly one constructed for use in transformation of a cell.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

A “plasmid” or a “plasmid vector” is a circular DNA molecule that can be introduced or transfected into bacterial or yeast cells by transformation, which plasmid will then replicate autonomously in the cell. A plasmid vector usually comprises a promoter sequence that is recognized by an RNA polymerase that may or may not be inherent to the host, which controls the expression of the desired gene, a heterologous nucleic acid operably linked to the promoter sequence, and a replication origin for increasing the copy number by induction with an exogenous factor. Plasmid replication origins are important because they determine plasmid copy number, which affects production yields. Plasmids that replicate to higher copy number can increase plasmid yield from a given volume of culture. (Suzuki et al., Genetic Analysis, p. 404, (1989). The promoter sequence contained in the plasmid vector, which sequence controls the expression of the desired gene, may be any promoter sequence capable of driving expression of the gene in that given host; i.e., promoter sequences recognized by particular RNA polymerases, e.g., those recognized by RNA polymerases derived from the T7, T3, SP6 and others such as LacZ, can be used. Promoters usable for this purpose include, but are not limited to, the lac, tip, tac, gal, ara and P.sub.L promoters etc. when Escherichia coli, is used so long as the above-described purpose is accomplished (Fitzwater, et al., Embo J. 7:3289-3297 (1988); Uhlin, et al., Mol. Gen. Genet. 165:167-179 (1979)). Furthermore, the plasmid vector may have a drug resistance gene used as a selection marker.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA or RNA, DNA or RNA that is a mixture of single- and double-stranded regions as well as hybrid molecules comprising a mixture of the above. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the gene-encoded amino acids. “Polypeptides” include amino acid sequences modified either by natural processes, such as posttranslational processing, or by chemical modification techniques that are well known in the art. Such modifications are well described in basic texts and in more detailed monographs; as well as in a voluminous research literature.

Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “reporter gene” refers to a gene that encodes a product that is detectable by standard methods, either directly or indirectly.

“Saccharomyces” generally refers to a yeast strain of the genus Saccharomyces cerevisiae, bakers yeast, is a unicellular microorganism that can exist as haploid or diploid forms, and reproduces by budding of daughter cells. Due to the ease of genetic manipulation of the S. cerevisiae genome, it has been extremely valuable in research efforts aimed at understanding basic biological phenomenon in eukaryotes. The genome of yeast has been completely sequenced and there is a wealth of information available with regards to the biology, genetics and molecular biology of this organism. In addition, well known and characterized tools for constitutive and inducible expression of heterologous proteins in yeast are available, which has made yeast a valuable tool for expression and purification of a host of therapeutic recombinant proteins. Furthermore, Saccharomyces yeast are widely used in the preparation of baked goods and vitamins, and in fermentation of alcoholic bevearages that are consumed by humans, which forms the basis of endowing yeast with the label of Generally Regarded As Safe (GRAS) for human consumption by the Food and Drug Administration.

In addition to being widely used in food and beverage preparation, yeast is part of the natural microflora resident in the human body. Resident strains of Saccharomyces cerevisiae have been isolated in healthy individuals from mucosal surfaces of the mouth and rectum. (See: Xu, J., C. M. Boyd, E. Livingston, W. Meyer, J. F. Madden, and T. G. Mitchell. 1999. Species and genotypic diversities and similarities of pathogenic yeasts colonizing women. J Clin.Microbiol. 37:3835-3843.)

Unlike the opportunistic microfloral yeast species, such as Candida albicans, which can lead to fatal infections in immuncompromised patients, resident Saccharomyces cerevisiae are rarely associated with such devastating health effects. In addition, it has been shown that administration of live yeast to healthy individuals and animal models does not lead to colonization and pathogenicity ( See: Maejima, K., K. Shimoda, C. Morita, T. Fujiwara, and T. Kitamura. 1980. Colonization and pathogenicity of Saccharomyces cerevisiae, MC16, in mice and cynomolgus monkeys after oral and intravenous administration Jpn.J Med.Sci.Biol. 33:271-276. See also Pecquet, S., D. Guillaumin, C. Tancrede, and A. Andremont. 1991. Kinetics of Saccharomyces cerevisiae elimination from the intestines of human volunteers and effect of this yeast on resistance to microbial colonization in gnotobiotic mice. Appl.Environ.Microbiol. 57:3049-3051. Non-limiting examples of Saccharomyces speicies sutiable for use in accordance with the teachings of the present invention include the group consisting of Saccharomyces cerevisiae, S. exiquus, S. telluris, S. dairensis., S. servazzii, S. unisporus, and S. kluyveri. The term “selectable marker gene” refers to a gene encoding a product that, when expressed, confers a selectable phenotype such as antibiotic resistance on a transformed cell.

With respect to “therapeutically effective amount” is an amount of the polynucleotide, antisense polynucleotide or protein, or fragment thereof, that when administered to a subject along with the bacterial fusant carrier, is effective to bring about a desired effect (e.g., an increase or decrease in a M-cell mediated immune response) within the subject.

“Transcriptional and translational” control sequences are DNA regulatory sequences, such as promoters, enhancers, polyadenylation signals, terminators, and the like, that provide for the expression of a coding sequence in a host cell.

A number of methods for delivering therapeutic formulations, including DNA expression constructs, into cells (e.g., E. coli cells) are known to those skilled in the art. A cell has been “transformed” or “transfected” or “transduced” by an exogenous or heterologous DNA or gene when such DNA has been introduced inside the cell. The transforming DNA may or may not be integrated (covalently linked) into the genome of the cell. In prokaryotes, bacteria and yeast cells for example, the transforming DNA may be maintained on an episomal element such as a plasmid or ligated into host DNA at specific restriction sites. As used herein, the term “transduction,” is used to describe the delivery of DNA to a cell using viral mediated delivery systems, such as, adenoviral, AAV, retroviral, or plasmid delivery gene transfer methods. As used herein the term, “transfection” is used to describe the delivery and introduction of a genetic element to a cell using non-viral mediated means, these methods include, e.g., calcium phosphate- or dextran sulfate-mediated transfection; electroporation; glass projectile targeting; and the like. These methods are known to those of skill in the art, with the exact compositions and execution being apparent in light of the present disclosure.

A “vector” is a replicon, such as plasmid, phage, or cosmid to which another nucleic acid segment may be operably inserted so as to bring about the replication or expression of the segment.

Modified Microflora Organims

Transforming of E. coli with plasmids is well known in the art. Introduction of polynucleotides into E. coli cells can be effected by methods described in many standard laboratory manuals, such as Davis et al., Basic Methods In Molecular Biology (1986) and Sambrook et al., Molecular Cloning: A Laboratory Manual, 2nd Ed., Cold Spring Harbor Laboratory Press, Cold Spring Harbor, N.Y. (1989) such as calcium phosphate transfection, DEAF-dextran mediated transfection, microinjection, cationic lipid-mediated transfection, electroporation, transduction, scrape loading, ballistic introduction or infection.

Transformation of LAB may be performed using a limited autolysis method as described in Leer et al. (WO095/35389), which is hereby incorporated by reference in its entirety. Transformation may also be performed on various LAB of interest according to methods and techniques disclosed in the following references, which are hereby incorporated by reference as if fully set forth herein. Published PCT application PCT/NL96/00409 discloses methods for screening non-pathogenic bacteria, in particular LAB of the genera Lactobacillus and Bifidobacterium, for the ability to adhere to specific mucosal receptors. An expression vector is also disclosed that comprises an expression promoter sequence, a nucleic acid sequence, and sequences permitting ribosome recognition and translation capability. This reference indicates that various strains of Lactobacillus can be transformed so as to express heterologous gene products including proteins of pathogenic bacteria. PCT/NL95/00135 describes a multicopy expression vector for use in Lactobacillus with a 5′non-translated nucleic acid sequence comprising at least the minimal sequence required for ribosome recognition and RNA stabilization, followed by a translation initiation codon. Further, oral administration of recombinant L. lactis has been used to elicit local IgA and/or serum IgG antibody responses to an expressed antigen (Wells et al., Antonie van Leeuwenhoek 1996 70:317-330). In addition, Casas et al. discloses in U.S. Pat. No. 6,100,388 that L. reuteri, can be transformed with heterologous DNA, and can express the foreign protein on the cell surface or secrete it, while EP 1084709 Al discloses the that Lactobacillus plantarum can, as well, be transformed to express an antigenic fragment either intracellularly or on the cell surface.

Methods for yeast transformation are also well known in that art. See for example co-pending U.S. patent application Ser. No. 10/280,769 filed Oct. 25, 2002 for additional details. See also “Guide to yeast genetics and molecular and cell biology” (2002) Edited by Christine Guthrie and Gerald Fink. These are two books in the Methods in Enzymology series. Volumes 350 and 351. Published by Academic Press and are herein incorporated by reference in their entirety.

According to one embodiment of the present invention, a generally regarded as safe (GRAS) microflora oragnaism, that is compatible with the mammalian body is modified by fusion with a second bacteria that harbors an expression system capable of expressing a protein in the LAB. In one particular embodiment the Lactic Acid Bacteria (LAB) that is compatible with a host body is from the genus Streptococcus or Lactococcus. In a preferred embodiment, the bacteria are from one of the following species Streptococcus thermophilus or Lactococcus lactis, however they may also be of the following species of Lactobacillus: lactobacillus: acidophilus, brevis; casei, delbrueckii, fermentum, or plantarum. In a particularly preferred embodiment, the preferred species are species that have been modified via mutation and/or selection that are more viable in the respiratory tract and can adhere preferentially to the mucous surfaces of the upper respiratory tract.

Several different bacteria with suitable expression systems can be fused with a non-pathogenic Streptococcus or Lactococcus bacteria to generate the desired modified LAB organism. In a preferred embodiment of the invention, Streptococcus or Lactococcus bacteria are fused with Escherichia coli (E. coli). Several different strains of E. coh that are commonly used for molecular cloning are HB101, C600, DH1, DH10B, DH5, α5 and β10. The strains mentioned are preferred because well-defined and commercially available expression systems for the production and expression of heterologous nucleic acids are already available for them.

In one embodiment of the invention, bacteria of one species are fused with bacteria of a different species. Two particular species of bacteria that have reported expression systems are Lactococcus lactis and Bacillus subtilis. Cocconcelli, P S, et al. “Single-stranded DNA plasmid, vector construction and cloning of Bacillus stearothermophilus alpha-amylase in Lactobacillus” Research in Microbiology 142(6): 643-52 (1991) and Kleerebezem, M., et al. “Controlled gene expression systems for lactic acid bacteria: transferable nisin-inducible expression cassettes for Lactococcus, Leuconostoc, and Lactobacillus spp.” Applied and Environmental Microbiology 63(11): 4581-84 (1997).

In one embodiment, the expression system of the present invention will contain a DNA construct comprising at least a nucleotide sequence encoding a desired antigen or therapeutic gene operably linked to a promoter that can direct expression of the heterologous sequence in a bacterial host. The polynucleotide encoding the antigenic or therapeutic fragment may include the coding sequence for the mature polypeptide or a fragment thereof, by itself or the coding sequence for the mature polypeptide or fragment in reading frame with other coding sequences, such as those encoding origin(s) of replication, an anchor, leader or secretory sequence, a pre-, or pro- or prepro-protein sequence, or other fusion peptide portions. For example, a marker sequence which facilitates selection of the fused polypeptide can be encoded. The polynucleotide may also contain non-coding 5′ and 3′ sequences, such as transcribed, non-translated sequences, splicing and polyadenylation signals, ribosome binding sites and sequences that stabilize mRNA.

In one embodiment, a LAB, such as the species thermophilus or lactis, is fused with E. coli in such a way as to allow the thermophilus or lactis bacteria to express an antigenic or therapeutic protein or polypeptide encoded by the E. coli associated DNA. Preferably, the antigenic polypeptide is capable of being expressed on the cell surface of the LAB-E. coli fusant, while the therapeutic protein is capable of being secreted. Hence, it is often advantageous to include an additional polynucleotide sequences coding for the amino-acid sequences which contain anchor, secretory or leader sequences, or additional sequences for stability during in vivo production. The protein of polypeptide fragments produced are then capable of either being expressed on the LAB-E. coli fusant's cell surface, or secreted, and thereby eliciting either an immune or therapeutic response.

Preferred polypeptide fragments include, for example, those coding for antigenic epitopes capable-of being recognized by the various immune initiating cells of the body, specifically, M cells, IgA and IgG cells, i.e., they are antigenic or immunogenic in an animal, especially in a human. Variants of the defined sequence and fragments also form part of the present invention. Preferred variants are those that vary from the referents by conservative amino acid substitutions. Other preferred fragments include biologically active, therapeutic fragments that mediate activity, including those with a similar activity or an improved activity, or with a decreased undesirable activity. Preferably, these polypeptide fragments retain the biological activity of the antigen or therapeutic, including antigenic activity.

Hence, in one particular embodiment the present invention relates to E. coli derived vectors that contain an antigenic or therapeutic polynucleotide or polynucleotides, host Streptococcus thermophilus or Lactococcus lactis cells that are genetically engineered by fusion with E. coli cell vectors, and to the production and expression of the encoded antigenic or therapeutic polypeptides by the host LAB cell-E. coli fusants. Suitable E. coli cells with appropriate expression systems can be purchased from various commercial sources, or genetically engineered, and made to incorporate expression systems or portions thereof for antigenic or therapeutic polynucleotides of the present invention.

Representative examples of appropriate LAB hosts for fusion with the E. coli cells and the in vivo production of antigenic and therapeutic proteins and/or polypeptides include Streptococcus thermophilus or Lactococcus lactis as well as Lactobacillus bacterial cells, such as: acidophilus, brevis, casei, delbrueckii, fermentum, or plantarum.

More particularly, the present invention includes recombinant E. coli vectors into which an antigenic and/or therapeutic construct comprising a DNA, cDNA or RNA sequence has been inserted, in a forward or reverse orientation. In a preferred aspect of this embodiment, the construct further comprises regulatory sequences, including, for example, a promoter, operably linked to the genetic sequence. Large numbers of suitable plasmids and promoters are known to those of skill in the art, and/or described below, and are also commercially available.

Hence, in one embodiment of the invention, the DNA construct will be a plasmid encoding at least an appropriate origin of replication for the desired bacterial host, a selectable marker gene and/or a reporter gene, a promoter operably linked to a heterologous nucleotide sequence encoding the antigen or therapeutic element fused to surface binding promoter or anchor region. The construct may also contain other suitable elements, such as transcription initiation sequences, secretion signal sequences and transcription termination sequences.

Plasmids will be chosen or created based on their ability to replicate in the host bacteria. Where the expression system is derived from E. coli, plasmid vectors into which the promoter and nucleotide sequence could be cloned include, for example pUC18, pUC19, pBR322, and pBluescript. For LAB appropriate plasmids include, for example, Lactococcus plasmids pAK80 or derivatives thereof, pTV32, pLTVI, pFXL03, pIC19H, pVA838 and pVA891. A plasmid from non-pathogenic Streptococcus is pER35. Plasmids from Lactococcus can be obtained from DSMZ, Braunschweig, Germany. Others have been described in the literature. In addition, plasmid vectors suitable for Lactococcus lactis are described in Geoffrey, M., et al., “Use of green fluorescent protein to tag lactic acid bacterium strains under development as live vaccine vectors” Applied and Environmental Microbiology 66(I): 383 (2000)). Plasmid vectors for Lactococcus lactis, Lactobacillus fermentum, and Lactobacillus sake are described in Piard, J., et al., “Cell wall anchoring of the Streptococcus pyogenes M6 protein in various lactic acid bacteria” Journal of Bacteriology 179(9): 3068-72 (1997). Some plasmid vectors are suitable for a wide range of Lactobacillus species, such as pPSC20 and pPSC22, described in Cocconcelli, P., et al., “Single-stranded DNA plasmid, vector construction and cloning of Bacillus stearothermophilus alpha-amylase in Lactobacillus” Res Microbiol 142(6): 643-52 (1991). Shuttle vectors, which are plasmids that are capable of expression in both of the parent bacteria used to create the fusant, could also be used. In this instance, appropriate shuttle vectors would contain origins of replication from both fusant species. Appropriate shuttle vectors for LAB include pFXL03, pWV01, pGKV210, pVA838 and pNZ123. Furthermore, E. coli and LAB shuttle vectors are described in Maassen, C., et al., Vaccine, ibid. and Bringel, et al. “Characterization, cloning, curing, and distribution in lactic acid bacteria of pLP1, a plasmid from Lactobacillus plantarum CCM 1904 and its use in shuttle vector construction” Plasmid 22(3): 193-202 (1989).

The plasmid could contain either selectable marker genes or reporter genes used to facilitate determining which bacteria contain the desired plasmid DNA. Possible selectable marker genes are antibiotic resistance markers, such as kan^{r}, tet^{r}, amp^{r} and the like. The gene for Beta galactosidase and the gene encoding green fluorescent protein (GFP) are examples of reporter genes. Alternatively, if the plasmid does not include a selectable marker or reporter gene the plasmid DNA could be detected in a variety of ways, such as, a dot blot using the plasmid DNA as a probe.

The choice of promoter will depend on the host bacteria and the antigen to be expressed. Promoters that could be used with E. coli expression systems include lambda PR, PL and Trp, as well as T3, T7, Gpt, SP6 and the lacZ promoter or Lac operon. Promoters for Lactococcus bacteria have been described in the literature. For example, one promoter that is well suited for both Lactobacillus plantarum and Lactococcus lactis and that has also been shown to be useful for expression in other LAB is the nisin inducible nisA promoter from Lactococcus lactis. See: deRuyter, P., et al., “Controlled gene expression systems for Lactococcus lactic with the food-grade inducer nisin” Appl. Environ. Microbiol. 62: 3662-67 (1996), Kleerebezem, M., “Controlled gene expression systems for lactic acid bacteria: transferable nisin-inducible expression cassettes for Lactococcus, Leuconostoc, and Lactobacillus spp.” Applied and Environmental Microbiology 63(11): 4581-84 (1997) and Geoffroy, M. et al., “Use of green fluorescent protein to tag lactic acid bacteria strains under development as live vaccine vectors,” Applied Environmental Microbiology 66(1):383-91 (2000). Other promoters may include the Lactococcus lactis MG1614 and MG1363 promoters, as well as the pH inducible and growth phase-dependent P170 promoter, and its variants, described in Madsen, S. M., et al., “Molecular Characterization Of The pH-Inducible And Growth Phase-Dependent Promoter P170 Of Lactococcus Lactis” Molecular Microbiology 32(1): 75-87 (1999). Further, a lactococcal promoter P₅₉ has been used in expression vectors of various Lactococcus lactis and Lactobacillus bacteria (Piard, J., et al., “Cell wall anchoring of the Streptococcus pyogenes M6 protein in various lactic acid bacteria” Journal of Bacteriology 179(9): 3068-72 (1997)). A useful promoter for Streptococcus thermophilus is the P25 promoter described in Geoffroy et al. In addition, the plasmid may contain multiple promoter sequences all operably linked to the sequence encoding the antigen. Each of the promoters in such a vector would be compatible with at least one of the parent bacteria used to make the fusant, furthermore, as mentioned the plasmid may contain multiple origins of replication, such as that from each parent species.

The nucleotide sequences encoding the antigen or therapeutic element and the surface binding promoter regions may be prepared in a variety of ways. These sequences can be obtained from any natural source or may be prepared synthetically using well-known DNA synthesis techniques. The sequences can then be incorporated into a plasmid, which is then used to transform the chosen bacterial host. Recently, advances in molecular biology with respect to recombinant production of proteins has made it possible to express foreign proteins at the outer surface of microorganisms by the technology called cell surface display. Sequences for surface binding promoter regions will be fused to the sequence of the antigen, such that the modified lactobacillus organism will present the antigen on its surface. Examples of such surface binding promoter regions are those used in the construct described in PCT/NL96/00135 and those described in Dieye, Y., et al., “Design of a protein-targeting system for lactic acid bacteria” Journal of Bacteriology 183(14); 4157-66 (2001).

One of the first surface-expression systems was developed in the mid 1980s by George P. Smith. He was able to express peptides or small proteins fused with pIII of the filamentous phage (see: Smith, G. P., Science, 228:1315-1317, 1985). Following that time, various systems of heterologous protein expression and secretion in microorganisms have been studied to develop new and better cell surface display and secretion systems by which proteins of interest can be expressed on the surface of the microorganisms or secreted. Using endogenous surface proteins, as a surface anchoring motifs, the current inventor has been studying the use of bacteria and yeast for the stable expression of proteins or peptides on the surface of a cell.

Bacteria, especially gram-negative bacteria such as E. coli, possess unique and complex cell envelope structures that may consist of an inner cellular membrane, periplasm, and outer cellular membrane. Hence, to efficiently transport foreign proteins to the cell surface a surface anchoring motif is needed. Therefore, in order to express a foreign peptide or protein, an appropriate bacterial surface protein has to be fused to the foreign protein of interest, at the genetic level, and the fusion protein expressed has to be transported through the inner cellular membrane and outer membrane to the surface of the bacteria where it then becomes anchored.

Given these factors, a surface anchoring motif needs to have several key characteristics. First of all, the surface protein to be used as an anchoring motif needs to have a sufficient secretion signal sequence motif to allow the transport of the foreign protein through the inner membrane of the cell. Secondly, a targeting signal for anchoring the foreign protein to the surface of the cell is also needed. Additionally, the overall fusion motif needs to have the capacity to not only accommodate foreign proteins or peptides of various sizes but to also express them in large amounts.

There are basically three groups of cell surface display systems that have been developed: C-terminal fusion, N-terminal fusion, and sandwich fusion. First of all, if a native surface protein has a discrete localization signal within its N-terminal portion, a C-terminal fusion motif may be used to fuse a foreign peptide to the C-terminal of that functional portion. For example, the Lpp-OmpA motif developed in E. coli uses a C-terminal fusion system (see: Georgiou, G., et al., Protein Eng., 9:239-247, 1996). Secondly, a N-terminal fusion motif has been developed which contains a C-terminal sorting signals to target foreign proteins to the cell wall. Examples of bacteria for which an N-terminal fusion motif has been developed include the Staphylococcus aureus protein A (see: Gunneriusson, E., et al., J. Bacteriol., 178:1341-1346, 1996), Staphylococcus aureus fibronectin binding protein B (see: Strauss, A., et al., Mol. Microbiol., 21:491-500, 1996), and Streptococcus pyogenes fibrillar M protein (see: Pozzi, G., et al., Infect. Immun., 60:1902-1907, 1992.). However, if the surface proteins do not have such anchoring regions the whole structure will be required for assembly. For this reason, a sandwich-fusion system has been developed, in which a foreign protein of interest is inserted into the surface protein motif. Several examples employing this system include E. coli PhoE (see: Agterberg, M., et al., Gene, 88:37-45, 1990), FimH (see: Pallesen, L., et al., Microbiology, '141:2839-2848, 1995), and PapA (see: Steidler, L., et al., J. Bacteriol., 175:7639-7643, 1993). Using these mechanisms, a person of ordinary skill in the art will be able to modify a given expression system for a given bacterium such as Streptococcus and Lactococcuss so as to effect the purposes of the present invention, namely expression, secretion and/or cell surface display of various antigenic and/or therapeutic elements.

For secretion of the translated protein into the extracellular environment, appropriate secretion signals may be incorporated into the desired polypeptide. These signals may be endogenous to the polypeptide or they may be heterologous signals. Hence, secretion signals may be used to facilitate delivery of the resulting protein. The coding sequence for the secretion peptide is operably linked to the 5′ end of the coding sequence for the protein, arid this hybrid nucleic acid molecule is inserted into a chosen plasmid adapted to express the protein in the host cell of choice. Plasmids specifically designed to express and secrete foreign proteins are available from commercial sources. For example, if expression and secretion is desired using an E. coli expression system, commonly used plasmids include pTrcPPA (Pharmacia); pPROK-C and pKK233-2 (Clontech); and pNH8a, pNH16a, pcDNAII and pAX (Stratagene), among others. Other secretion signal systems are those such as the M6 preprotein from Streptococcus pyrogens described in Dieye, Y., et al., “Design of a protein-targeting system for lactic acid bacteria” Journal of Bacteriology 183(14); 4157-66 (2001) and those set forth, such as SP13, SP10, SP307 and SP310 recognized by signal peptidase I or II, in Ravn, P., et al., “The Development Of TnINuc And Its Use For The Isolation Of Novel Secretion Signals In Lactococcus Lactis” Gene 242: 347-356 (2000).

Hence, in one embodiment the invention embodies methods for producing heterologous proteins in a host organism whereby the protein is processed through the secretory pathway of the host. Secretion is achieved by transforming a host organism, i.e., E. coli, with a plasmid containing a DNA construct comprising a transcriptional promoter operably linked to DNA sequences encoding a secretion signal peptide, for instance the portion of the BAR1 C-terminal domain or the Staphylococcus aureus protein A that is capable of directing the export of heterologous proteins or polypeptides.

Examples of other various secretion systems described for use in E. coli include U.S. Pat. No. 4,336,336 (filed Jan. 12, 1979); European Pat. Application Publication Numbers 184,169 (published Jun. 11, 1986), 177,343 (published Apr. 9, 1986) and 121,352 (published Oct. 10, 1984); Oka, T. et al. (1985); Gray, G. L. et al. (1985); Ghrayeb, J. et al. (1984) and Silhavy, T. et al. (1983). For the most part, these systems make use of the finding that a short (15-30) amino acid sequence present at the amino NH₂-terminus of certain bacterial proteins, which proteins are normally exported by cells to noncytoplasmic locations, are useful in similarly exporting heterologous proteins to noncytoplasmic locations. These short amino acid sequences are commonly referred to as “signal sequences” as they signal the transport of proteins from the cytoplasm to noncytoplasmic locations. In Gram-negative bacteria, such noncytoplasmic locations include the inner membrane, periplasmic space, cell wall and outer membrane. At some point just prior to or during transport of proteins out of the cytoplasm, the signal sequence is typically removed by peptide cleavage thereby leaving a mature protein at the desired noncytoplasmic location. Site-specific removal of the signal sequence, also referred to herein as accurate processing of the signal sequence, is a preferred event if the correct protein is to be delivered to the desired noncytoplasmic location.

Hence, in one embodiment the present invention relates to a Streptococcus thermophilus or Lactococcus lactis organism that is modified by fusion with an E. coli bacteria that contains a plasmid encoding a heterologous nucleic acid that is operably linked to a promoter capable of driving expression of the genetic element in the modified host bacteria. According to one particular embodiment, the heterologous nucleic acid is polynucleotide sequence coding for an antigen that is either capable of being secreted or displayed on the cell surface of the bacteria. In either case, the plasmid encoding the heterologous nucleic acid will also contain, the appropriate secretion or anchor sequence information required for either secretion or cell surface delivery and expression. According to this embodiment, the protein or peptide fragment produced within the fusant comprises an antigen capable of eliciting an immune response when it comes into contact with an immune related cell of the body.

In the case wherein the protein is secreted, the related immune cell is expected to be a secreted IgA antibody, however, it is also likely that the secreted antigenic fragment may be endocytosed by the M cells of the Peyer's patches, in which case the antigenic protein or fragment may come into contact with the various components of the M cell pocket, including CTLs, B cells, macrophages and dendritic cells, thereby inducing a mucosal immune response. In the case where the protein or antigenic fragment is anchored and displayed on the cell surface of the fusant, the antigenic fragment may come into direct contact with the cell surface membrane of the M cells thereby directly interacting with the various components of the M cell directly to illicit a mucosal immune response.

According to another particular embodiment, the heterologous nucleic acid is polynucleotide sequence coding for a therapeutic protein or peptide fragment that is either capable of being secreted or displayed on the cell surface of the bacteria. In either case, the plasmid encoding the heterologous genetic element will also contain the appropriate secretion or anchor sequence information required for either secretion or cell surface delivery and expression. According to this embodiment, the protein or peptide fragment produced within the fusant comprises a therapeutic such that when it is expressed it produces a protein or fragment thereof necessary for modifying and or correcting a diseased state. Particularly, the heterologous nucleic acid encodes a protein capable of being secreted into the lumen of the respiratory tract, such as insulin, whereby when the protein is secreted it is capable of being absorbed and modifying a diseased state, such as diabetes.

M Cell Training

In one embodiment of this invention, the modified microflora organisms will be targeted to M cells, such as those associated with Peyer's patches in the nasal-associated lymphoid tissue (NALT) in the respiratory system. M cell targeting can be accomplished in a variety of ways, including using compounds that bind to M cell surface compounds. Such compounds include polypeptides, such as M cell receptors or surface antigens, carbohydrates, and glycoconjugates. M cell targeting may involve compounds that specifically bind to M cells as well as compounds that specifically bind to cells of tissue with which M cells are associated, such as the epithelial cells of the upper respiratory tract.

One example of a compound that binds to M cells are adhesins from bacteria and viruses that target M cells, such as the Yersinia species and Salmonella typhi, respectively. (Clark, M. A., et al., “M-cell surface β1 integrin expression and invasin-mediated targeting of Yersinia pseudotuberculosis to mouse Peyer's patch M cells” Infect Immun. 66:1237-43 (1998); Baumler, A. et al., “The Ipf fimbrial operon mediates adhesion of Samonella typhirium to murine Peyer's patches” Proc. Natl. Acad. Sci. USA 93: 279-83 (1996). Such bacterial and viral adhesins are proteins that mediate M cell binding. Furthermore, the σI protein of the reovirus has also been used to target M cells. Wu, Y., et al., “M cell-targeted DNA vaccination” Proc. Natl Acad. Sci. USA 98(16): 9318-23 (2001). Hence, in one particular embodiment, the plasmid containing the heterologous nucleic acid and secretion or anchor signal also contains a polynucleotide sequence coding for the reovirus σ 1 protein fused to the sequence coding for the heterologous nucleic acid. In another embodiment, the polynucleotide sequence coding for the reovirus σ 1 protein is contained within a separate plasmid.

Another compound that binds specifically to M cells is lectin. M cell targeting of lectin bearing-liposomes to M cells using various types of lectin is described in U.S. Pat. No. 6,060,082. In one embodiment of the present invention, the regeneration step in the fusion process includes the addition of lectins wherein when the outer cellular membrane is reformed around the fusant, lectins, which are capable of targeting M cells are incorporated into the membrane surface. Furthermore, lectin, derivatized to a lipid (Avanti Polar lipids), could also be incorporated into the cell wall during bacterial growth and reproduction, if added to the culture media.

Antibodies that bind specifically to M cell surface proteins such as receptors or surface antigens may also be used for M cell targeting. Antibodies to such surface proteins can be generated in a variety of ways that are well known in the art, using the entire protein of interest (either the precursor or the processed protein) or a portion thereof.

The M cell targeting compounds described above can be incorporated into the cell wall of the modified microflora. This can be accomplished by adding the M cell targeting compound to modified lactobacillus protoplasts that are regenerating cell walls. In a preferred embodiment, the M-cell targeting compound will be derivatized to lipids designed to act as membrane anchors. Such functionalized lipids can be purchased from Avanti Polar Lipids, Inc. (Alabaster, Ala.).

Alternatively, a plasmid in the modified microflora could encode an M cell targeting polypeptide. In one embodiment the plasmid containing the sequence for the antigen would also contain the sequence for the M cell targeting polypeptide. In this embodiment, the M cell targeting polypeptide could be attached to the sequence for the antigen. Alternatively, the M cell targeting polypeptide sequence could be attached to surface binding promoter regions and operably linked to a promoter region, such that expression of the plasmid would produce two heterologous proteins.

In another embodiment, a second plasmid would contain the M cell targeting polypeptide sequence attached to surface binding promoting regions and operably linked to a promoter, such that the parent bacteria would harbor two different recombinant plasmids.

In an additional embodiment, the plasmid containing the heterologous nucleic acid may also contain the polynucleotide sequence coding for a synthetic peptide containing an a integrin-binding motif (arginine-glycine-aspartic acid, RGD) fused to the sequence coding for the heterologous nucleic acid, for the enhancement delivery. It has been shown that integrin proteins are capable of binding the RGD motif are located on the apical surface of a polarized human bronchial epithelial cells. Scott, E. S., et al., “Enhanced Gene Delivery To Human Airway Epithelial Cells Using An Integrin-Targeting Lipoplex” The Journal Of Gene Medicine 3: 125-134 (2001). Receptor-ligand interaction is between peptides containing the RGD (arginine-glycine-aspartic acid) motif and several members of the integrin family of cell surface receptors have been well-characterized. Hence, in this approach receptor-mediated endocytosis is used to gain entry to the target epithelial cells. Scott, E. S., et al., “Enhanced Gene Delivery To Human Airway Epithelial Cells Using An Integrin-Targeting Lipoplex” The Journal Of Gene Medicine 3: 125-134 (2001) and also, Hart, S., et al., “Gene Delivery And Expression Mediated By An Integrin-Binding Peptide” Gene Ther. 2: 552-554 (1995).

Upon administration, which is preferably intranasal, the modified microflora will be capable of settling in and/or colonizing at least part of the respiratory tract, such as the mouth, the throat, the larynx, and/or the lungs, or a combination thereof. According to one preferred embodiment, the microflora is such that it mainly settles in the upper respiratory tract, although the invention is not limited thereto, at which time the said host will be displaying or secreting the antigenic or therapeutic elements encoded therein allowing them to come into contact with the mucosal cells of the gut, according to the invention. The antigens expressed and/or therapeutics delivered by the host thus can come into contact with the mucosal layer, the lining and/or the wall of the g. i. tract and more specifically with M cells within said wall that can mediate an immune response against the antigen(s) thus presented to the macrophages, dendritic cells, B-lymphocytes and/or CTL cells of the M cell pocket. This immunological response by the cells within the wall of the g. i. tract constitutes a significant immune response as defined above, and it acts as a trigger for a further systemic immunological reaction/response in the body of the human or animal to which the vaccine has been administered, which magnifies the significance of the response and increases the bodies subsequent protective mechanisms.

It is within the scope of that present invention that the modified microflora will preferably exhibit a persistence with in the respiratory system of the individual to be immunized, upon intranasal administration, preferably exceeding 3-9 days, more preferably greater than 15 or even 20 days, although this is not required. Selection of a host strain based on a given phenotype, particularly the ability to survive within and/or cling to a given cell type, such as the M cells of the upper respiratory tract, for a prolonged period of time, is well within the abilities of one of ordinary skill in the biological.

The skilled person will be able to select appropriate microflora to be modified in accordance with the teachings of the present invention having one or more of the following properties: stability of the construct encoding the antigen or therapeutic in the bacterial or yeast selected; level of expression of the antigen or therapeutic in or by the microflora organism; regulation of expression of the heterologous protein, site of expression of the antigen or therapeutic; stability of antigen produced; as well as the biochemical properties of the strain used, including but not limited to its sugar fermentation profile, cell wall composition, structure LTA, structure pepticloglycan, 16S RNA sequence, acid resistance, bile acid resistance, agglutination properties, adjuvanticity, immune modulating properties, in vitro adherence properties, mannose-specific adherence, presence of proteinaceous adherence factors, presence of mapA-like adherence factors, presence of large proteinaceous adherence factors with repeated amino acid sequences; and the interaction of the microflora organism with cells of the individual to which the organiskm is to be administered (i.e. as part of a vaccine according to the invention) including but not limited to its persistence (which is preferably as defined above), viability, in vivo expression of antigen or therapeutic and/or tissue-specific persistence.

The foregoing is intended to be illustrative of the embodiments of the present invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein.

### EXAMPLES

The following description sets forth the general procedures involved in practicing the present invention. To the extent that specific materials are mentioned, it is merely for purposes of illustration and is not intended to limit the invention. Unless other-wise specified, general cloning procedures, such as those set forth in Sambrook et al., Molecular Cloning, supra or Ausubel et al. (eds) Current Protocols in Molecular Biology, John Wiley & Sons (2000) (hereinafter “Ausubel et al.”) are used. Accordingly, the following examples illustrate how one skilled in the art may make use of the current invention to produce a modified organism derived from either Streptococcus thermophilus and/or Lactococcus lactis bactria that expresses a heterologous antigen. Further, these examples show how one may use the modified organism to invoke an immune response in a mammal. Methods in molecular biology, cell biology, and immunohistochemistry that are not explicitly described in this disclosure have already been amply reported in the scientific literature.

### Example 1

Production of a Modified Lactococcus Organism

Selection of Bacteria and Cloning of the Plasmid DNA

The modified Lactococcus organism will be formed through the fusion of Lactococcus with a second bacteria that contains a recombinant plasmid. In this example, Lactococcus lactis (ATCC #7962) will be fused with E. coli HB101 (ATCC #33694).

The E. coli HB 101 will contain a recombinant plasmid, pSYG3 that encodes GFPuv, which is a GFP variant that has been optimized for bacterial expression (Crameri, A., et al. “Improved green fluorescent protein by molecular evolution using DNA shuffling” Nat. Biotechnol. 14: 315-19 (1996)). GFPuv has been optimized for maximal fluorescence when excited by UV light (360-400 nm) and can be amplified from pBAD-GFPuv (Clontech, Palo Alto, Calif.) using the following primers: CAT GCA TGC CAT GGC TAG CM AGG AGA AGA AC and CCG GGT ACC GAG CTC GAA TTC (SEQ. ID. NO. 1) (Geoffroy, M., et al., “Use of green fluorescent protein to tag lactic acid bacterium strains under development as live vaccine vectors” Applied and Environmental Microbiology 66(1): 383-91 (2000)).

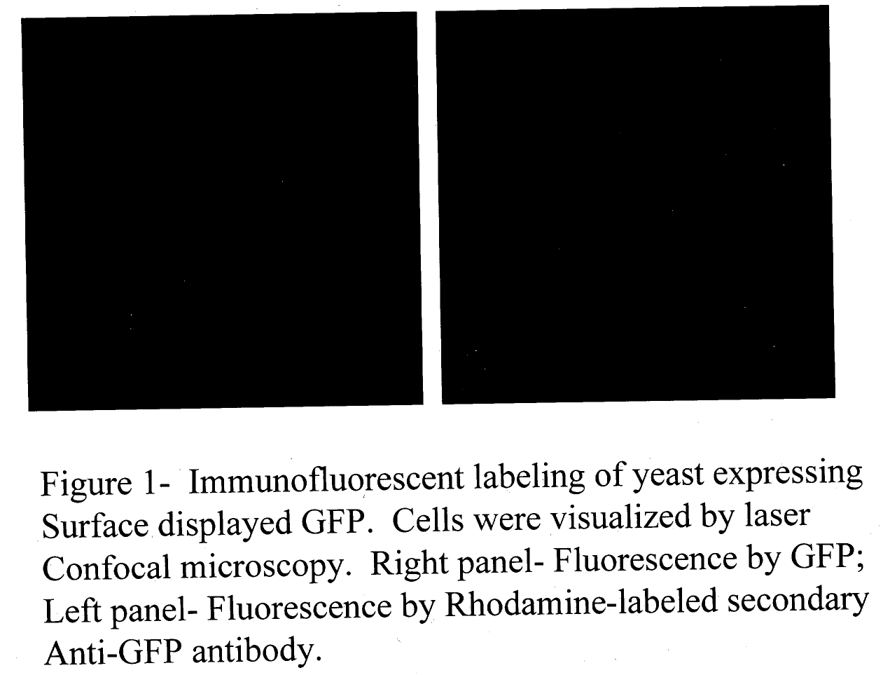

PSYG3 will be constructed from pUC19 and will include the origin of replication from pBR322, a kanamycin resistance gene, and a T7 promoter sequence that is operably linked to a nucleotide sequence encoding GFPuv fused with surface binding promoter regions. The surface binding promoter regions may be sequences for the signal peptide from the lactococcal Usp45 preprotein and for the cell wall anchor domain from the M6 preproprotein of Streptococcus pyogenes along with the necessary transcriptional terminators. The signal peptide sequence will be upstream from the GFPuv sequence while the cell wall anchor domain will be downstream from the GFPuv sequence. For details, see Deite, Y., et al., “Design of a protein-targeting system for lactic acid bacteria” Journal of Bacteriology 183(14): 4157-66 (2001). Also see FIG. 1 for a map of pSYG3. Cloning of the plasmid, transformation of the E. coli cells with the plasmid, and selection of colonies containing the plasmid will be accomplished according to procedures that are well known to one of ordinary skill in the art as set forth in references such as Sambrook, et al., Molecular Cloning: A Laboratory Manual, 3^{rd} edition (Cold Spring Harbor Press, Cold Spring Harbor, N.Y.) (2001) and Ausubel, et al., Current Protocols in Molecular Biology, (Wiley, N.Y.) (2001).

Alternatively, the plasmid may also contain other DNA sequences, such as a sequence encoding the sigma 1 protein of reovirus operably linked to a T7 promoter in addition to surface binding promoter regions, such as those described above. Expression of such a protein would accomplish M-cell targeting.

Formation of Escherichia Coli and Lactococcus Protoplasts

Protoplasts of both bacterial strains may be formed using the following methods. Lactococcus lactis cells will be grown in MRS media (Difco) at 26 C. until the exponential growth phase has been reached. E. coli HB 101 harboring pSYG3 will be grown in LB at 37 C. for until the exponential growth phase has been reached. Then, chloramphenicol will be added to the E. coli culture and pSYG3 selectively amplified for 16 hours. After centrifugation of the cultures at 2000×g for 30 minutes, the resulting cell pellets will be washed and resuspended in a hypertonic solution (0.01 M Tris hydrochloride [pH 7.5], 0.3-0.5 M mannitol) that contains lysozyme (20 ug/ml) and incubated at room temperature for 5-15 minutes. An aliquot of the resulting protoplasts will be gently overlaid on plates with the appropriate regeneration media (MRS or LB) and colony formation observed to insure the protoplasts are able to regenerate cell walls. Protoplasts must be maintained in the hypertonic solution, which may contain sucrose instead of mannitol, until they regenerate cell walls, to prevent lysis due to osmotic pressure.

Fusion of E. Coli and L. Lactis Protoplasts

To fuse the protoplasts, 1×10⁹−10×10¹⁰ E. coli protoplasts in the hypertonic solution described above may be added to 0.5-1 ml of the L. lactis protoplasts 1×10⁹-10×10⁹ in the same hypertonic solution. 0.5 m1-1.5 ml of 20%-70% PVA or PEG will be added to the mixture, and the solution will be gently agitated to achieve thorough mixing. The mixture may be incubated for 1-30 minutes at room temperature, and protoplast aggregation and fusion monitored by phase-contrast microscopy. When cell growth reaches an exponential stage, the protoplasts will be washed three times and diluted in 3-7 ml of the hypertonic solution used above. A small amount of the resulting solution (0.5-2 ml) will be plated on MRS agar with kanamycin and incubated at 26 C.

The MRS agar will select for L. lactis and modified L. lactis, replica on a minimum medium and/or an ELISA test can be performed with antiserum against LAB. LAB strains identification will also be performed on the tomato agar plates. The kanamycin will select for bacteria containing pSYG3. Thus, the resulting colonies will be modified 1. acidophilus fusants harboring pSYG3. Alternatively, because GFP is also a reporter gene, colonies containing pSYG3 may be selected based on green fluorescence under ultraviolet light.

### Example 2

Characterization of the Phenotype of the Modified Lactococcus Organism

Various assays will be performed to confirm that the desired modified lactobacillus organism has been generated. Single colonies will be 1) picked from the selective plates described above, 2) grown in MRS broth, and 3) replated on MRS agar with kanamycin. Steps 1-3 will be repeated five times to obtain purified colonies.

Tests to determine the physiological properties of the modified lactobacillus organism will be performed according to the instruction manual from the API ZYM and API 20A biochemical test systems. The characteristics of the parent bacteria are set forth in Holt, et al., Bergey's Manual of Determinative Bacteriology 9^{th} ed. (Williams & Wilkins, Baltimore, Md.) (1994), which is a comprehensive guide that allows identification of bacteria that have been described and cultured.

Based on the selective pressures described above, the modified Lactococcus organism should have a phenotype corresponding to that of the genus Lactococcus. Therefore, the cells should be spherical and Gram positive. In liquid media, the cells will occur in pairs or in short chains. They should require a complex media for growth, and their metabolism should be fermentative, producing L(+)-lactic acid without gas. In addition, the cells should be catalase negative and oxidase negative.

The modified Lactococuss organism should not have a phenotype corresponding to the genus Escherichia. Some of the above tests for lactobacillus will also show that the modified lactobacillus organism is not Escherichia, as Escherichia cells reduce nitrates, are gram negative, and are catalase positive.

### Example 3

Characterization of the Genotype of the Modified Lactococcus Organism

Southern blots will be performed to determine whether the modified Lactococcus organism has the expected genotype. Chromosomal DNA will be extracted according to standard procedures. See Saito and Miura. Plasmid DNA preparation and Southern hybridization will be performed as described in Sambrook, Molecular Cloning: A Laboratory Manual.

Chromosomal DNA from the parent bacteria and plasmid DNA will be used as probes. Low homology would be observed if Lactococcus lactis chromosomal DNA were probed with E. coli chromosomal DNA or if E. coli were probed with L. lactis DNA. In contrast, the L. lactis and E. coli chromosomal DNA probes will share 50% or greater homology with the modified Lactococcus chromosomal DNA, as the fusant should contain chromosomal DNA from both of the parent bacteria. One of skill of the art would also appreciate that if the two parents are more closely related and therefore have highly homologous chromosomal DNA, as may occur in some embodiments of this invention, the difference in the degree of hybridization that occurs between the chromosomal DNA of the two parents and the degree of hybridization that occurs between the parent and fusant chromosomal DNA will be less dramatic than that described in this example. In such cases, one may rely more heavily on identification of the plasmid DNA through Southern hybridization to characterize the genotype of the fusant.

### Example 4

Assays to Determine Expression of Antigen in the Modified Lactococcus Organism Ex Vivo

Detection of GFP Fluorescence

Expression of GFP fluorescence in the modified Lactococcus organism may be examined in several ways, according to known procedures. As noted above, plates with the modified Lactococcus organism may be photographed under UV illumination to identify colonies that are expressing GFP. In addition, GFP production in modified Lactococcus cells suspended in PBS may be observed using epifluorescence microscopy. Photographs of such observations using appropriate film may be taken. Finally, GFP expression may be measured by preparing modified Lactococcus cell lysates and assaying for fluorescence using a fluorimeter.

Western Blots Performed on Total Protein Extracts and Cell Fractions to Localize GFP Expression

Western blots of total protein extracts and various fractions of the cell will be performed to test for expression of GFPuv and to show that GFP is being targeted to the cell membrane. Total protein extracts will be prepared according to well-known procedures set forth in references such as Ausubel, et al., Current Protocols in Molecular Biology. Cell fractionation will be performed according to the method outlined in Piard, J. -C., et al. “Cell wall anchoring of the Streptococcus pyogenes M6 protein in various lactic acid bacteria.” Briefly, 2 nil of exponential-phase culture may be microcentrifuged for 5 minutes at 4 C. at 4,300 g. The resulting cell pellet and supernatant will be separated and concentrated. Proteins in the supernatant will be precipitated using trichloroacetic acid (TCA). The cell pellet will be resuspended in TES, treated with lysozyme, and the resulting protoplasts centrifuged at low speed. The supernatant will contain proteins released from the cell wall, which will be precipitated using TCA. Proteins will then be extracted from the protoplast pellet as described in Dieye, Y., et al. “Design of protein-targeting system for lactic acid bacteria” Journal of Bacteriology 183(14): 4157-66.

Total protein and cell fraction samples may then be analyzed by Western blot using rabbit GFP antiserum (Invitrogen) as the primary antibody and horseradish peroxidase conjugated anti rabbit antisera (Sigma) as the secondary antibody and for detection. A known amount of recombinant GFPuv (Clontech) will be run as a control. The amount of GFPuv on the Western blots may be estimated by scanning them and comparing the signals from the control and experimental lanes. Western blotting is described in detail in Sambrook, et al., Molecular Cloning: A Laboratory Manual.

### Example 5

Assays to Determine Expression of and Immune Response to GFPUV In Vivo

Mice may be immunized intranasally. Various regimens may be used to produce optimal results. For example, groups of 6 BALB/c mice will be immunized on days 1 or 1-3 and then at 28 days with the modified lactobacillus organisms described above or with Lactococcus-E. coli fusants that are identical to those described above except that they do not harbor plasmids. An alternative immunization protocol would be immunization at 7-day intervals on days 0, 7, 14, and 28.

Assays for Expression of GFP In Vivo

Expression of GFP in vivo and its uptake by tissue associated with M cells will be assayed in several ways. With intranasal administration, mice may be sacrificed after 8-12 hours and cells harvested by performing a bronchoalveolar wash on each mouse. The cells will then be centrifuged, washed twice with PBS, and resuspended in PBS. The suspension may be stained with an acidotropic probe, such as Lyso Tracker Red (Molecular Probes, Eugene, Oreg.), which binds organelles and fluoresces at 590 nm, and then observed under an epifluorescent microscope to detect GFPuv. See Geoffrey, 1Vl., et al. Applied and Environmental Biology 66(1): 383.

Alternatively, mice may be sacrificed after 8-12 hours and nasal-associated lymphoid tissue (NALT) and its flanking tissue harvested. Tissue will then be fixed in formalin, embedded in paraffin, and thin sections observed under a fluorescent microscope to detect GFP. GFP may also be detected by incubating the thin tissue sections with the rabbit anti GFP antibody and the hydrogen-peroxidase conjugated antirabbit antisera described above and then detecting GFP by adding diaminobenzidene. M cells may be visualized with FITC-labeled Ulex europaeus agglutinin 1 (Vector Laboratories).

Assays for Immune Response to GFP

To test for an immune response to GFP, blood samples may be taken before the primary dose, and at two weeks, four weeks, and eight weeks after the primary dose. Immune response will be evaluated using Enzyme-Linked Immunosorbent Assay (ELISA). Briefly, microtiter plates will be coated overnight with 100 ng per well of GFPuv (Clontech, Palo Alto, Calif.) in PBS and then serum samples will be applied to the plates and incubated for two hours at room temperature. Horseradish peroxidase conjugated anti mouse antisera will be used for detection.

In addition, lymphocyte proliferation in response to exposure to GFPuv will be measured. Ten days following immunization lymphocytes will be isolated and incubated in multi-well plates for 72 hours in medium alone or in medium containing GFPuv. ³H thymidine will be added to the cultures for the last 18 hours of incubation and its uptake measured using a liquid scintillation counter.

### Example 6

Delivering HbsAg Antigen and IL-2 Gene

HBV surface antigen genes Pre-S2 and S will be obtained by PCR amplification from plasmid pEco63 (ATCC31518). Mouse IL-2 gene fragment will be obtained by PCR from plasmid pMUT-1 (ATCC37553). Both genes will be placed under Lac-Z promoter in fusion or under a separate T7 promoter in pUC18. The genes may also be cloned in a shuttle vector. Plasmid containing only pre-S2/S gene is named pPS2S. The plasmid with both pre-S2/S and IL-2 genes is named pPS2S/IL2. (Chow et al. J Vir. January 1997: 169-178). The two genes may also be cloned into another shuttle vector in a fusion or under a separate promoter. The DNA will be transformed into E. coli DH5a and or HB 101. Plasmid DNA will then be amplified in E. coli cultures. Exponentially grown E. coli will be protoplasted as described above and fused with Lactococcus lactis. Fusants will be selectively grown on LAB MRC plate and tomato juice plate and or a synthetic medium (Broach et al. Gene. 8(1979)121-133.). Selection will be made for the expression plasmid via Kanamycin along with a transgene product assay, as following.

The HBsAg protein in the fusant medium broth or cell pellets will be assayed by the AUSZYME Monoclonal antibody kit (Abbott Lab). The intracellular protein should be released by a Ten-Brock ground bead homogenizer. Membrane bound proteins should be released by treatment with Triton X-100. Production of the antigen should be found up to 3% of total cellular protein. IL-2 activity will then be tested by a proliferated assay (Chow et al) and a ELISA using anti-IL-2 antibody.(Pharmigen).

BALB/c and C57b1/6 mice will be immunized with 1-10×10⁹ cfu of LAB of up to 3 doses. Serum will be collected by tail bleeding beginning from day 2. HbsAg antibodies will determined using serological assays known in the art and/or detailed in the present specification.

### Example 7

Construction of pYD1-Based Plasmids

pYD1 is a galactose-inducible expression vector purchased from Invitrogen, which directs expression of proteins on the yeast cell wall. The antigens of interest, VP7, HA and NA were PCR amplified using the primers listed in Table 1. The resulting PCR products were cloned into either the BamHI/EcoRI (VP7) or the BamHI/XbaI (NA and HA) sites of pYD1.

### Example 8

Construction of pGPD-DSPLY and it's Derivatives

pGPD-DSPLY functions as a target vector for constitutive expression of a number of proteins displayed on the cell wall. Names and sequences of PCR primers used to construct pGPD-DSPLY and it's derivatives are listed in Table 1. pGPD-DSPLY contains sequences encoding the leader sequence of yeast α-mating factor and the cell-wall anchoring domain (C-terminal 350 amino acids) of Saccharomycse cerevisiae α-agglutinin. First, sequences encoding the α-leader peptide followed by two amino acid spacers (Gly and Ala) were PCR amplified from the yeast chromosome (strain S288C) using primers BamLALPHAfwd and EcoLALPHArev and cloned into BamHI and EcoRI sites of p426GPD (described in Mumberg et al., 1995, Yeast vectors for the controlled expression of heterologous proteins in different genetic backgrounds, Gene 156: 119-122) to construct pSecY. Next, sequences encoding the cell-wall anchoring domain of α-agglutinin was PCR amplified from yeast chromosomal DNA (strain S288C), using the oligonucleotides Agglfwd and Agglrev, and cloned into the ClaI/XhoI sites of p426GPD to obtain pGPDAnch. pGPD-DSPLY was constructed by subcloning an EcoRI/XhoI fragment containing α-agglutinin sequences into the same sites of pSecY.

Vectors for surface display of antigens NA, VP7 (pNADSPLY, pVP7DSPLY) were constructed as follows: NA and VP7 encoding sequences were PCR amplified from a cloned copy of these gene using primer pairs NAnewfwd/NAnewrev and VP7newfwd/VP7newrev, respectively, and cloned upstream of α-agglutinin sequences into the EcoRI/HindIII sites of pGPDAnch to obtain pNAAnch and pVP7Anch. Next, an EcoRI/XhoI fragment from pNAAnch and pVP7Anch were subcloned into the same sites of pSecY to obtain pNADSPLY and pVP7DSPLY, respectively. To verify correct positioning of antigens to the cells wall, pGFPDSPLY was constructed basically as described above; GFP encoding sequences were PCR amplified from plasmid pQB125-fPA (Qbiogene) using primers sgGFPfwd and sgGFPrev.

Construction of an HA surface display vector pHADSPLY was performed by cloning PCR-amplified HA sequences into the EcoRI/HindIII sites of pGPDDSPLY. Due to the presence of an EcoRI site within HA ecoding sequences, a sticky end PCR strategy was used (Zheng, G., Sticky-end PCR: new method for subcloning. 1998, Biotechniques, 25: 206-208) to facilitate the cloning. First, two separate HA amplification reactions were performed using primer pairs HAfwd1/Hanewrev and HAfwd2/HAnewrev. After digestion with DpnI (to remove background plasmid) and HindIII, equal molar amounts of the two PCR products were mixed, heat denatured, allowed to cool to room temperature, and cloned into the EcoRI/HindIII sites of pGPDDSPLY.

In order to facilitate immunological detection of the antigens, sequences encoding various epitope tags (His₆ and HA) were cloned into the EcoRI sites of pNADSPLY and pVP7DSPLY vectors which positions the tag in between the antigen-encoding, and the cell-wall anchoring sequences. The oligonucleotides used for these constructions are listed in Table 1.

### Example 9

Preparation of Lactobacillus Surface Display Vectors

Genes expressing antigens of interest were cloned into SfiI/AscI sites of the surface display vector pSC111AE. As a result of this construction, VP7, HA, NA and GFP are fused N-terminally to the secretion signal of the amylase gene and C-terminally to the cell-wall anchoring domain of the prtp protease. The expression of the fusion proteins is driven by the constitutively active XyI promoter. The sequences of oligonucleotides used for PCR amplification of the various antigens are shown in Table 1.

TABLE 1

SEQ ID NO for oligonucleotides used for construction of surface display
expression vectors

| | | | |
|---|---|---|---|
| | | | Target |
| SEQ ID | | | vector or |
| NO. | Oligonucleotide | Sequence | purpose |

| | | | |
|---|---|---|---|
| 2 | VP7-1 | 5′-CGGGATCCGGTGGCCAGAACTATGGACTTAATATAC-3′ | pYD-1 |
| 3 | VP7-2 | 5′-CCGGAATTCTTAATTTATCCCATCAACGAC-3′ | pYD-1 |
| 4 | HA-1 | 5′-CGGGATCCGGTGGTGGTGACACAATATTATAGGC-3′ | pYD-1 |
| 5 | HA-2 | 5′-CCGGAATTCTTAGATGCATATTCTGCAC -3′ | pYD-1 |
| 6 | NA-1 | 5′-CGGGATCCGGIGGTGGTCATTCAATTCAAACTGG-3′ | pYD-1 |
| 7 | NA-2 | 5′-CCGGAATTCTTACTTGTCAATGGTGAA -3′ | pYD-1 |
| 8 | BamLALPHAfwd | 5′-CCGGATCCATGAGATTTCCTTCAATTTTTAC-3′ | p426GPD |
| 9 | EcoLALPHArev | 5′-GCGAATTCAGCACCTCTTTTATCCAAAGATACC-3′ | p426GPD |
| 10 | Agglfwd | 5′-CCATCGATGGTTCTGCTAGCGCCAAAAGCTC-3′ | p426GPD |
| 11 | Agglrev | 5′-CAGCTCGAGTTAGAATAGCAGGTACGAC-3′ | p426GPD |
| 12 | HAfwd1 | 5′-AA1TCGACACAATATGTATAGGCTAC-3′ | pGPDAnch |
| 13 | HAfwd2 | 5′-CGACACAATATGTATAGGCTAC-3′ | pGPDAnch |
| 14 | HAnewrev | 5′-ACCAAGCTTGATGCATATTCTGCAC-3′ | pGPDAnch |
| 15 | NAnewfwd | 5′-CGGAATTCCATTCAATTCAAACTGGAAG-3′ | pGPDAnch |
| 16 | NAnewrev | 5′-ACCAAGCTTCTTGTCAATGGTGAATGG-3′ | pGPDAnch |
| 17 | VP7newfwd | 5′-CGGAATTCCAGAACTATGGACTTAATATAC-3′ | pGPDAnch |
| 18 | VP7newrev | 5′-ACCAAGCTTATTTAICCCATCAACGAC-3′ | pGPDAnch |
| 19 | sgGFPfwd | 5′-CGGAATTCATGGCTAGCAAAGGAGAAG-3′ | pGPDAnch |
| 20 | sgGFPrev | 5′-GGAAGCTTATCGATGTTGTACAGTTC-3′ | pGPDAnch |
| 21 | HAECOfwd | 5′-AATTTTACCCATACGACGTCCCAGATTACGCTGGTGCCG-3′ | epitope TAG |
| 22 | HAECOrev | 5′-AATTCGGCACCAGCGTAAACTGGGACGTCGTATGGGTAA-3′ | epitope TAG |
| 23 | HISECOfwd | 5′-AATTTCATCACCATCACCATCACGGTGCCG-3′ | epitope TAG |
| 24 | HISECOrev | 5′-AATTCGGCACCGTGATGGTGATGGTGATGA-3′ | epitope TAG |
| 25 | GfpSfilForward | 5′-TAGGCCCAGCCGGCCGCCGCTAGCAAAGGAGAAGAACTCT | pSc111AE |
| | | TCACTGG-3′ | |
| 26 | GFPAsclReverse | 5′-AAGGCGCGCCATCGATGTTGTACAGTTCATC-3′ | pSC111AE |
| 27 | Vp7SfilForward | 5′-TAGGCCCAGCCGGCCGCCCAGAACTATGGACTTAATATAC-3′ | pSc111AE |
| 28 | Vp7AscIReverse | 5′-AAGGCGCGCCATTTATCCCATCAACGAC-3′ | pSc111AE |
| 29 | HASfilForward | 5′-TAGGCCCAGCCGGCCGCCGACACAATATGTATAGGCTAC-3′ | pSC111AE |
| 30 | HAAsclReverse | 5′-AAGGCGCGCCGATGCATATTCTGCACTGC-3′ | pSC111AE |
| 31 | NASfilForward | 5′-TAGGCCCAGCCGGCCGCCCATTCAATTCAAACTGGAAGTC-3′ | pSC111AE |
| 32 | NAAsclReverse | 5′-AAGGCGGGCCCTTGTCAATGGTGAATGG-3′ | pSc111AE |

### Example 10

Expression of Proteins on Yeast Cell Surface

pYD1-based expression-EBY 100 yeast transformed with pYD1 or pYD1-based expression vectors were grown overnight at 30° C. in YNB-CAA medium containing 2% glucose. Cells were harvested by centrifugation and resuspended in YNB-CAA medium containing 2% galactose to an OD₆₀₀ of 0.5˜1. Cells were grown at 20˜25° C., and samples were harvested at regular time intervals to analyze for expression by immunofluorescent staining.

pGPD-DSPLY-based expression-W303-1A cells transformed with pGDP-DSPLY or it's derivatives were grown to mid-log phase at 30° C. in Synthetic drop out medium without uracil. Cell were harvested and analyzed for protein expression as described below.

### Example 11

Detection of Antigens on Yeast Cell Surface

Detection of antigens on yeast cell surface was accomplished by immunofluorescence labeling of whole cells followed by confocal microscopy.

An exponentially growing culture of yeast was fixed by addition of 1/10^{th} volume of formaldehye to the culture medium, with continued incubation of the shaking culture for 1 Hour. The fixed cells were washed with PBS three times and incubated with a monoclonal anti-GFP antibody for 1.5 hrs at room temperature (RT). After washing with PBS, the cells were incubated for 1 hr at RT with the secondary antibody conjugated with Rhodamine. Cells were washed with PBS, mounted on a microscope slide and visualized with confocal microscopy. As shown in FIG. 1, GFP was expressed on the surface of yeast cells as indicated by the pattern of the cellular distribution of GFP-associated fluorescence. In addition, a similar pattern of GFP distribution was detected by immunofluorescence analysis of yeast cells expressing surface-displayed GFP.

### Example 12

Protocol for Immunization of Animals with Recombinant Yeast

Six weeks old female Balb/c mice were inoculated by oral, intranasal or subcutaneous routes with yeast displaying VP7, HA or NA on the cell surface. Booster inoculations were performed every two weeks. Mice were inoculated with either yeast expressing surface-displayed antigen or yeast containing empty vector. Three different routes of inoculation were used: oral, intranasal or subcutaneous. The number of mice used for each experiment is outlined in Table 2. Blood samples were collected before the first vaccination and every two weeks there after. Mice were sacrificed after 8-weeks, and trachea, lung and intestine washings were collected. The presence of antigen-specific IgG and IgA antibodies in the blood and tissue samples were detected by ELISA.

A. Vaccine Preparation

For the galactose-inducible expression (pYD1), yeast cells expressing virus antigens VP7, HA or NA, and cells containing empty vector were grown in YNB-CAA medium and induced for expression with 2% galactose. For constitutive expression (pGPD-DSPLY), yeast cells were grown to mid-log phase in synthetic defined (SD) dropout media without uracil. Cells were harvested at mid-log phase, washed with and resuspended in PBS to a concentration of 5×10⁹/ml.

B. Vaccination

Oral: 0.1 ml (5×10⁸)/mice

Intra-nasal: 0.02 ml (1×10⁸)/mice

Subcutaneous: 0.1 ml (5×10⁸) mixed with 0.1 ml adjuvant/mice (complete Freund's adjuvant for the first Subcutaneous inoculation, incomplete Freund's adjuvant for booster). The first inoculation was done on week zero. Booster inoculations were done at weeks two, four and six with the same amount of cells.

### Example 13

Measurement of Antibody Response

Blood samples (˜0.1 ml) were taken from the eye bowl. Serum were separated by centrifugation, and stored at −20° C. The Lung and intestines were separated from the sacrificed animal and washed with PBS. The tissue washings were collected into Eppendorf tubes and centrifuged. The supernatants were stored at −20° C.

The samples were tested by ELISA for the presence of antigen-specific antibodies. The Viral antigens, VP7, HA or NA were coated on 96 well plates. After blocking of non-specific binding sites, samples of sera, lung or intestine washings were diluted with PBS and added to each well. Horseradish peroxidase-labeled secondary antibodies (anti-IgG or anti-IgA) were used to detect antibody-antigen complexes.

Tables 3, 4, 5 and 6 below show the raw data from each vaccination protocol. Table 3 shows serum antibody titer for yeast Flu vaccine using pGPD Table 4 shows serum antibody titer for yeast rotavirus vaccine using pGPD Table 5 shows serum antibody titer for yeast Flu vaccine using pYD1 Table 5. Serum antibody titer for yeast Flu vaccine using pYD and Table 6 shows serum antibody titer for yeast rotavirus vaccine for pYD1.

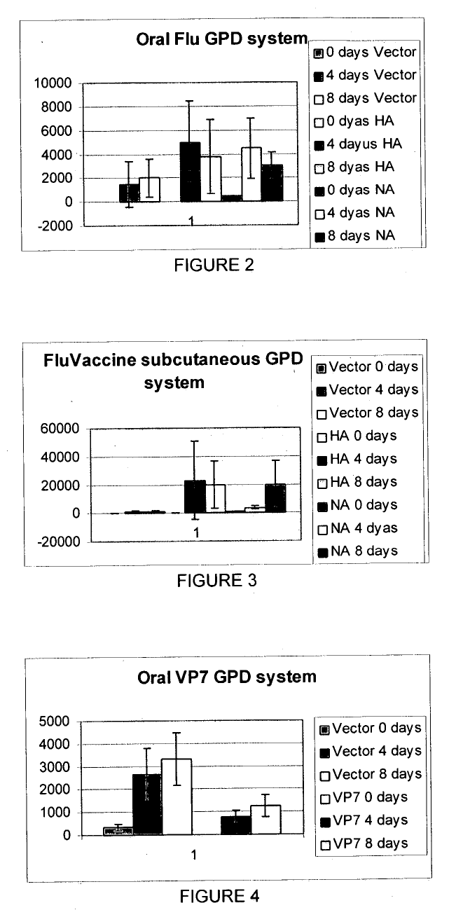

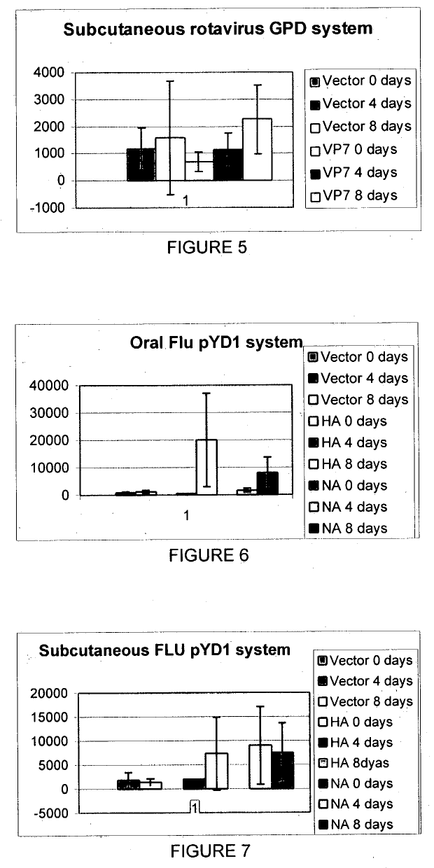

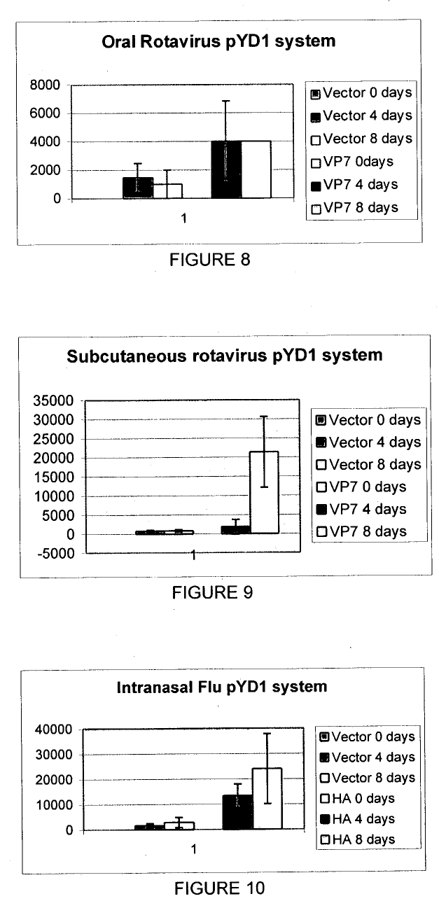

FIGS. 2-10 graphically depict the data presented in Tables 3-6. As can been seen, when compared to the plasmid controls, each immunogenic composition of the present invention successfully elicited an immune response in the test animal. 2A and 2B, subcutaneous injection of recombinant yeast induced a humoral antibody response (IgG production). In addition, intranasal delivery of recombinant yeast led to induction of both a humoral (IgG production, FIG. 2C) and a mucosal (IgA production, FIG. 2D) immune response.

TABLE 2
Number of animals in each experimental group

| | A | A1 | A2 | A3 | B | B1 | B2 | B3 |
|---|---|---|---|---|---|---|---|---|
| Vaccine | control | VP7 | HA | NA | control | VP7 | HA | NA |
| Oral | 4 | 4 | 4 | 4 | 4 | 4 | 4 | 4 |
| Intra- | 4 | 4 | 4 | 4 | 4 | 4 | 4 | 4 |
| nasal | | | | | | | | |
| sub- | 4 | 4 | 4 | 4 | 4 | 4 | 4 | 4 |
| cuta- | | | | | | | | |
| neous | | | | | | | | |

Note:
A, pYD1 system;
B, pGPD-DSPLY system.

TABLE 3
Serum antibody titer for yeast Flu vaccine using pGPD

| Vaccine | | pGPD | | | pGPD-HA | | | PGPD-NA | | |
|---|---|---|---|---|---|---|---|---|---|---|
| weeks | | 0 | 4 | 8 | 0 | 4 | 8 | 0 | 4 | 8 |
| Oral | 1 | 0 | 0 | 2000 | 0 | 2000 | 8000 | 500 | 2000 | 2000 |
| | 2 | 0 | 2000 | 2000 | 0 | 8000 | 2000 | 500 | 4000 | 2000 |
| | 3 | 0 | 4000 | 4000 | 0 | 8000 | 4000 | 500 | 8000 | 4000 |
| | 4 | 0 | 0 | 0 | 0 | 2000 | 1000 | 500 | 4000 | 4000 |
| | Mean | <500 | 1500 | 2000 | <500 | 5000 | 3750 | 500 | 4500 | 3000 |
| | SD | 0 | 1915 | 1633 | 0 | 3464 | 3096 | 0 | 2517 | 1155 |
| SQ | 1 | 500 | 2000 | 2000 | 500 | 4000 | N/A | 500 | 4000 | N/A |
| 2 | 250 | N/A | N/A | 250 | 64000 | N/A | 1000 | 4000 | 32000 | |
| | 3 | 500 | 1000 | 500 | 250 | 16000 | 32000 | 500 | N/A | N/A |
| | 4 | 500 | 1000 | 1000 | 500 | 8000 | 8000 | 1000 | 2000 | 8000 |
| | Mean | 438 | 1333 | 1167 | 375 | 23000 | 20000 | 750 | 3333 | 20000 |
| | SD | 125 | 577 | 764 | 144 | 27785 | 16971 | 289 | 1155 | 16971 |

SQ = Subcutaneous

TABLE 4
Serum antibody titer for yeast rotavirus vaccine using pGPD

| Vaccine | | pGPD | | | pGPD-VP7 | | |
|---|---|---|---|---|---|---|---|
| weeks | | 0 | 4 | 8 | 0 | 4 | 8 |
| Oral | 1 | 500 | 2000 | 4000 | 0 | 500 | 1000 |
| | 2 | 250 | 2000 | 2000 | 0 | 1000 | 2000 |
| | 3 | 250 | 4000 | 4000 | 0 | 500 | 1000 |
| | 4 | N/A | N/A | N/A | 0 | 1000 | 1000 |
| | Mean | 333 | 2667 | 3333 | <500 | 750 | 1250 |
| | SD | 144 | 1155 | 1155 | 0 | 289 | 500 |
| SQ | 1 | 0 | 2000 | 250 | 500 | 1000 | 4000 |
| | 2 | 0 | N/A | N/A | 1000 | 2000 | 2000 |
| | 3 | 0 | 1000 | 4000 | 250 | 500 | 2000 |
| | 4 | 0 | 500 | 500 | 1000 | 1000 | 1000 |
| | Mean | <500 | 1167 | 1583 | 688 | 1125 | 2250 |
| | SD | 0 | 764 | 2097 | 375 | 629 | 1258 |

N/A means Not Available Serum antibody titer for yeast Flu vaccine

TABLE 5
Serum antibody titer for yeast Flu vaccine using pYD1

| Vaccine | | pYD1 | | | pYD1-HA | | | pYD1-NA | | |
|---|---|---|---|---|---|---|---|---|---|---|
| weeks | | 0 | 4 | 8 | 0 | 4 | 8 | 0 | 4 | 8 |
| Oral | 1 | 0 | 1000 | 2000 | 0 | N/A | N/A | 0 | 2000 | 16000 |
| | 2 | 0 | 500 | 1000 | 0 | 500 | 32000 | 0 | 500 | 4000 |
| | 3 | 0 | 1000 | 1000 | 0 | N/A | N/A | 0 | 2000 | 8000 |
| | 4 | 0 | 1000 | 500 | 0 | 500 | 8000 | 0 | 2000 | 4000 |
| | Mean | 0 | 875 | 1125 | 0 | 500 | 20000 | 0 | 1625 | 8000 |
| | SD | 0 | 250 | 629 | 0 | 0 | 16970 | 0 | 750 | 5656 |
| IN | 1 | 0 | 2000 | 500 | 0 | 16000 | 8000 | 0 | 1000 | 8000 |
| | 2 | 0 | 2000 | 4000 | 0 | 8000 | 32000 | 0 | N/A | N/A |
| | 3 | 0 | 500 | 4000 | 0 | 16000 | 32000 | 0 | N/A | N/A |
| | 4 | 0 | 2000 | N/A | 0 | N/A | N/A | 0 | N/A | N/A |
| | Mean | 0 | 1625 | 2833 | 0 | 13333 | 24000 | 0 | 1000 | 8000 |
| | SD | 0 | 750 | 2020 | 0 | 4618 | 13856 | 0 | N/A | N/A |
| SQ | 1 | 0 | 2000 | 500 | 0 | 2000 | 16000 | 0 | 2000 | 2000 |
| | 2 | 0 | 4000 | 2000 | 0 | 2000 | 2000 | 0 | 16000 | 16000 |
| | 3 | 0 | 1000 | 1000 | 0 | 2000 | 4000 | 0 | 16000 | 4000 |
| | 4 | 0 | 250 | 2000 | 0 | 2000 | N/A | 0 | 2000 | 8000 |
| | Mean | 0 | 1812 | 1375 | 0 | 2000 | 7333 | 0 | 9000 | 7500 |
| | SD | 0 | 1625 | 750 | 0 | 0 | 7572 | 0 | 8082 | 6191 |

SQ = Subcutaneous

TABLE 6
Serum antibody titer for yeast rotavirus vaccine for pYD1

| Vaccine | | pYD1 | | | pYD1-VP7 | | |
|---|---|---|---|---|---|---|---|
| weeks | | 0 | 4 | 8 | 0 | 4 | 8 |
| Oral | 1 | 0 | 2000 | 2000 | 0 | 4000 | 4000 |
| | 2 | 0 | 0 2000 | 0 | 2000 | 4000 | |
| | 3 | 0 | 2000 | 0 | 0 | >2000 | 4000 |
| | 4 | 0 | 2000 | 0 | 0 | 8000 | |
| | Mean | 0 | 1750 2000 | 0 | 3500 | 4000 | |
| | SD | | | | | | |
| IN | 1 | | | | 0 | 1000 | 2000 |
| | 2 | | | | 0 | 500 | 1000 |
| | 3 | | | | 0 | 500 | 1000 |
| | 4 | | | | 0 | 1000 | 4000 |
| | Mean | | | | 0 | 750 | 2000 |
| | SD | | | | | | |
| SQ | 1 | 0 | 500 | 1000 | 0 | 500 | 32000 |
| | 2 | 0 | 1000 | 1000 | 0 | 1000 | 16000 |
| | 3 | 0 | 500 | 500 | 0 | 4000 | 16000 |
| | 4 | 0 | 1000 | 0 | | | |
| | Mean | 0 | 688 | 750 | 0 | 1833 | 21333 |
| | SD | | | | | | |

### Administration

For intranasal administration the composition may be in a formulation suitable for intranasal administration in the form of aerosols or insufflations for intratracheobronchial administration; and the like. Preparations of such formulations are well known to those skilled in the pharmaceutical arts. See for instance, Guillaume, C., et al., “Aerosolization of Cationic Lipid-DNA Complexes: Lipoplex Characterization and Optimization of Aerosol Delivery Conditions” Biochemical and Biophysical Research Communications 286: 464-471 (2001). For instance, use of ultrasonic nebulization can be used to aerosolize a formulation containing the modified microflora, which can than be delivered intranasally by a metered dose inhaler (MDI). MDIs are small, portable devices that deliver medication in an aerosol form to be inhaled. The medication, in this case a formulation containing the modified microflora, is dissolved or suspended in a liquid contained in a small canister. The canister fits into a plastic device, the mouth-piece, that releases a set amount of medication, or a metered dose.

Ideally, nebulizer-generated particles are small and form a string like composition composed of little, cubic units. The use of an aerosol route for antigenic/therapeutic delivery relies upon two requirements: the ability to get a formulated concentration of the antigenic/therapeutic in to the lung tract while at the same time reducing systematic side-effects. Therapeutic efficacy will, therefore, depend on the effective penetration of the microflora formulation inside the lungs; this penetration relies upon the parameters of aerosol kinetics governed by the physico-characteristics of the complex of concern, the equipment used for aerosolization and inhalation conditions. Lung deposition will thus depend on the mean mass aerodynamic diameter (MMAD) of nebulized particles. See Pascal, S., et al., “Antibiotherapie En Aerosols” Revue Maladies Respiratoires 9: 145-153 (1992). One advantage of delivering LAB carriers of an antigen or therapeutic over current methods involving the delivery of naked DNA or DNA conjugated with a target delivery protein and/or encapsulated in lipids is that plasmid DNA is known to be degraded by shear stresses, such as those present in an ultrasonic nebulizer, by delivering the heterologous nucleic acid within a LAB fusant carrier this complication is greatly avoided.

According to one embodiment of the invention, modified microflora may be formulated in a lipoplex preparation as a dry powder. The lipid formulation can be sonicated and mixed with the modified microflora and incubated at room temperature with or without a saline solution of 50 mM NaCl for 30 min or 1 h before use. The use of sodium chloride (NaCl) at a concentration above or equal to 50 mM may result in a higher level of transfection in cultured cells. Aerosol may be generated using the DP 10 ultrasonic nebulizer (Air Medica, France) and ideally the solution will have a total volume of 4 ml and contain 400 μg of modified mircoflora and lipids in various amounts. To minimize loss of sample due to splattering, the flow of air through the nebulizer should be restricted to an appropriate level. The size spectrum of various solutions generated from the DP 10 ultrasonic nebulizer can then be characterized by using a PALAS PCS2000 optical counter, a lipid-to-pDNA ratio of 0.8 (w:w) is optimal.

A basic parameter in aerosolization is the size-distribution of nebulized particles; the final size and number of particles generated depend on the nebulized product and on the type of nebulizer used. Ideally, the ultrasonic nebulizer-generated particles should have a diameter within 1 and 2 μm and form a polydispersed aerosol. They should be in a size-range suitable for reaching the deep airways, which provides the lipoplexes with a more marked therapeutic capability. That is the sonic nebulization should be capable of being performed in such a way that they will allow one to: (i) prepare highly concentrated and stable complexes quickly aerosolized while avoiding flocculation, (ii) preserve the integrity and activity of the complex made, (iii) obtain the right size for particle aerosolization and inhalation to enable their deposition into the lungs.

Hence, the vaccine preparation may be in the form of a powder, such as a freeze dried powder that is reconstituted before use, e.g., using a suitable liquid; or in the form of a solid or liquid preparation that is mixed with solid, semi-solid or liquid food prior to administration. The dosage and method of administration can be tailored to achieve optimal efficacy and will depend on factors that those skilled in the medical arts will recognize.

The effective amount of the antigenic or therapeutic composition to be given to a particular patient will depend on a variety of factors, several of which will be different from patient to patient. A competent clinician will be able to determine an effective amount of a antigenic or therapeutic composition to administer to a patient to elicit an appropriate immune or therapeutic response. Dosage of the composition will depend on the type of treatment, route of administration, the nature of the antigens or therapeutics, calculated absorption rates for the therapeutics, etc. Utilizing LD₅₀ animal data, and other information available for ingestion and or absorption via the respiratory system, a clinician can determine the maximum safe dose for an individual, depending on the route of administration. Utilizing ordinary skill, the competent clinician will be able to optimize the dosage of a particular therapeutic composition in the course of routine clinical trials.

With respect to an effective amount, the amount of modified microflora administered is not particularly critical, so long as it is an amount that will allow the yeast and/or bacteria to settle into and colonize the upper respiratory tract, preferably within the Peyer's patches and/or to cause a significant immune response. A suitable amount will be at least 10⁵ cfu, preferably 10¹⁰-10¹² cfu per dose, which allows a sufficient amount of bacteria to pass the gut into the intestine.

Once properly compounded in accordance with the teachings of the present invention, the microflora compostions of the present invention can be used to elicit immune responses and provide heterologous nucleic acids to the intestinal mucosa of a wide range of animals including, but not limited to primates, goats, cattle, horses, birds, fish, pigs, rats, mice cats and dogs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety. In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for inducing an immune response in an animal comprising:
providing an immunogenic composition formulated for intranasal administration to said animal wherein said immunogenic composition comprises a microflora organism having an expression vector wherein said expression vector comprises a heterologous nucleic acid that encodes for an antigen.

2. The method for inducing an immune response in an animal according to claim 1 wherein said microflora organism is a yeast or bacteria.

3 The method for inducing an immune response in an animal according to claim 1 wherein said antigen is selected from the group consisting of tumors, bacteria, viruses, parasites, and fungi.

4. The method for inducing an immune response in an animal according to claim 3 wherein said viruses are selected from the group consisting of influenza, hepatitis, HIV, and rotavirus.

5. The method for inducing an immune response in an animal according to claim 2 wherein said yeast in is selected from the group consisting of Saccharomyces cerevisiae, S. exiquus, S. telluris, S. dairensis, S. servazzii, S. unisporus, and S. kluyveri.

6. The method for inducing an immune response in an animal according to claim 2 wherein said bacteria in is selected from the group consisting of Bifidobacterium sp, Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

7. The method for inducing an immune response in an animal according to claim 1 wherein said intranasal formulation is selected from the group consisting of powder, a freeze dried powder a liquid preparation, a semi-solid, yogurt milk and cheese.

8. A method for inducing an immune response in an animal comprising:
providing an intranasal formulation of transformed yeast wherein said yeast comprise a heterologous nucleic acid encoding for an antigen where in said antigen is expressed on the surface of said yeast.

9. The method for inducing an immune response in an animal according to claim 8 wherein said yeast is Saccharomyces cerevisiae.

10. The method for inducing an immune response in an animal according to claim 8 wherein said antigen is derived from a virus.

11. A method for inducing an immune response in an animal comprising:
providing an intranasal formulation of transformed Saccharomyces cerevisiae wherein said transformed Saccharomyces cerevisiae comprises a heterologous nucleic acid encoding for an immunoprotective epitope from influenza A.

12. A method for inducing an immune response in an animal according to claim 11 wherein said immunoprotective epitope is influenza HA or NA.

13. An immunogenic composition comprising:
an intranasal formulation of a microflora organism having an expression vector wherein said expression vector comprises a heterologous nucleic acid that encodes for an antigen.

14. The immunogenic composition comprising according to claim 13 wherein said microflora organism is a yeast or bacteria.

15 The immunogenic composition comprising according to claim 13 wherein said antigen is selected from the group consisting of tumors, bacteria, viruses, parasites, and fungi.

16. The immunogenic composition comprising according to claim 15 wherein said viruses are selected from the group consisting of influenza, hepatitis, HIV, and rotavirus.

17. The immunogenic composition comprising according to claim 14 wherein said yeast in is selected from the group consisting of Saccharomyces cerevisiae, S. exiquus, S. telluris, S. dairensis, S. servazzii, S. unisporus, and S. kluyveri.

18. The immunogenic composition comprising according to claim 14 wherein said bacteria in is selected from the group consisting of Bifidobacterium sp, Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

19. The immunogenic composition comprising according to claim 13 wherein said intranasal formulation is selected from the group consisting of aerosols, drops, snuffs, suppositories and creams.

20. An immunogenic composition comprising:
an intranasal formulation of transformed yeast wherein said yeast comprise a heterologous nucleic acid encoding for an antigen where in said antigen is expressed on the surface of said yeast.

21. The immunogenic composition comprising according to claim 20 wherein said yeast is Saccharomyces cerevisiae.

22. The immunogenic composition comprising according to claim 20 wherein said antigen is derived from a virus.

23. An immunogenic composition comprising:
an intranasal formulation of transformed Saccharomyces cerevisiae wherein said transformed Saccharomyces cerevisiae comprises a heterologous nucleic acid encoding for an immunoprotective epitope from influenza virus.

24. The immunogenic composition comprising according to claim 23 wherein said immunoprotective epitope is inflenza HA or NA.

25 The immunogenic composition comprising according to claim 18 wherein said bacteria is fused with an E. coli.

26 The immunogenic composition comprising according to claim 25 wherein said E. coli is selected from the group consisting of HB101, C600, DH1, DHa5 and P10.

27. The immunogenic composition comprising according to claim 25 wherein said E. coli comprises a plasmid.

28 The immunogenic composition comprising according to claim 27 wherein said plasmid comprises a heterologous nucleic acid operably linked to a promoter capable of driving expression of said heterologous nucleic acid in a host organism.

29. The immunogenic composition comprising according to claim 28, wherein said heterologous nucleic acid codes for an antigen.

30. The immunogenic composition comprising according to claim 29, wherein said antigen is expressed on said bacteria's cell surface.

31. The immunogenic composition comprising according to claim 29, wherein said antigen is secreted.

32. The immunogenic composition comprising according to claim 13 or 29, wherein said antigen is selected from the group consisting of Mycobacterium leprae antigens, Mycobacterium tuberculosis antigens, Rickettsia antigens, Chlamydia antigens, Coxiella antigens, malaria sporozoite and merozoite protein antigens, the circumsporozoite protein antigen from Plasmodium berghei sporozoites, diphtheria toxoids, tetanus toxoids, Clostridium antigens, Leishmania antigens, Salmonella antigens, E. coli antigens, Listeria antigens, Borrelia antigens, the OspA and OspB antigens of Borrelia burgdorferi, Franciscella antigens, Yersinia antigens, Mycobacterium africanum antigens, Mycobacterium intracellular antigens, Mycrobacterium avium antigens, Treponema antigens, Schistosome antigens, Filaria antigens, Pertussis antigens, Staphylococcus antigens, Hemophilus antigens, Streptococcus antigens, the M protein of S. pyogenes, pneumococcus antigens, Shigella antigens, Neisseria antigens, anthrax toxin, clostridium, staphylococcus, helicobacter, peudomona, yersinia, rabies virus, salmonella and pneumonia.

33. The immunogenic composition comprising according to claim 13 or 29, wherein said antigen is selected from the group consisting of mumps virus antigens, hepatitis virus a.b.c.d.e. HBV antigens, Herpes virus antigens, parainfluenza virus antigens, rabies antigens, polio virus antigens, Rift Valley Fever virus antigens, dengue virus antigens, measles virus antigens, rotavirus antigens, Human Immunodeficiency Virus (HIV) antigens, the gag, pol, and env protein antigens, gp 120 and gp 160 of the HIV env, respiratory syncytial virus (RSV) antigens, snake venom antigens, human tumor antigens, Vibrio cholera antigens, HCV, HAV, HPV, TB, Herpes, rubella, influenza, poliomyelitis, rotavirus, surface glycoprotein of malaria parasite, Epstein barr virus, poxvirus, rabies virus, CEA and cancer antigens.
